(19) United States
(12) Patent Application Publication
(10) Pub. No.: US 2013/0273171 A1
(43) Pub. Date: Oct. 17, 2013

(54) ORAL FORMULATION FOR DEXLANSOPRAZOLE

(71) Applicants: Zhiqun Shen, Hangzhou (CN); Yu Zhang, Hangzhou (CN); Enjun Fu, Hangzhou (CN); MIn Michael He, Ellicott City, MD (US); Fang-yu Liu, Union City, CA (US)

(72) Inventors: Zhiqun Shen, Hangzhou (CN); Yu Zhang, Hangzhou (CN); Enjun Fu, Hangzhou (CN); MIn Michael He, Ellicott City, MD (US); Fang-yu Liu, Union City, CA (US)

(73) Assignee: HANDA PHARMACEUTICALS, LLC, Fremont, CA (US)

(21) Appl. No.: 13/510,924

(22) PCT Filed: Nov. 18, 2010
(86) PCT No.: PCT/US10/57280

§ 371 Date: May 18, 2012

Related U.S. Application Data
(60) Provisional application No. 61/263,274, filed on Nov. 20, 2009.

(51) Int. Cl. A61K 31/4439

(52) U.S. Cl. CPC A61K 31/4439
USPC 424/499; 514/338; 264/12; 427/2.1

(57) ABSTRACT

A stable formulation of dexlansoprazole for treating a digestive disorder, and methods of manufacturing the same.

### RELATED APPLICATIONS

This application claims priority to U.S. Provisional Application No. 61/263,274, filed Nov. 20, 2009 the disclosure of which is incorporated herein by reference in its entirety, where permitted.

### FIELD OF THE INVENTION

The present invention generally relates to the field of pharmaceutical sciences. More specifically, the present invention relates to pharmaceutical formulations containing benzimidazole proton pump inhibitors, such as lansoprazole and dexlansoprazole, methods for preparing such formulations, and the use of specific formulations for the treatment of digestive disorders.

### BACKGROUND

The following includes information that may be useful in understanding the present embodiments. It is not an admission that any of the information provided herein is prior art, or relevant, to the presently described or claimed embodiments, or that any publication or document that is specifically or implicitly referenced is prior art.

Dexlansoprazole solids often possess low and variable stability, which can lead to difficulties in preparing pharmaceutically acceptable formulations. As such, developing stable solid formulations containing dexlansoprazole remains an ongoing challenge.

### DESCRIPTION OF THE RELATED ART

KAPIDEX™ is a commercially available formulation containing dexlansoprazole. PREVACID™ is a commercially available formulation containing lansoprazole.

### SUMMARY OF THE INVENTION

Some embodiments provide a process for preparing a dexlansoprazole composition comprising: preparing a first mixture by mixing dexlansoprazole, a base, a sugar alcohol, and a first excipient in an organic solvent; and drying to provide a dexlansoprazole composition. In some embodiments, the process for preparing a dexlansoprazole composition further comprises: layering the first mixture on a support matrix, by spraying the first mixture onto the support matrix to provide a coated excipient mixture, wherein the drying comprises drying the coated excipient mixture to provide a dexlansoprazole composition. In some embodiments, the process for preparing a dexlansoprazole composition further comprises: preparing a second mixture by mixing a second excipient and support matrix; and layering the first mixture on the second mixture, by spraying the first mixture onto the second mixture to provide a coated excipient mixture, wherein the drying comprises drying the coated excipient mixture to provide a dexlansoprazole composition.

In some embodiments, concerning the process for preparing a dexlansoprazole composition, the base is selected from the group consisting of Ca(OH)₂, CaO, a mixture of CaCO₃ and NaOH, and mixtures thereof. In some embodiments, concerning the process for preparing a dexlansoprazole composition, the base is Ca(OH)₂. In some embodiments, concerning the process for preparing a dexlansoprazole composition, the base does not include a component selected from the group consisting of MgO and MgCO₃. In some embodiments, concerning the process for preparing a dexlansoprazole composition, the base does not include a Mg²⁺ counterion. In some embodiments, concerning the process for preparing a dexlansoprazole composition, the base includes a Ca²⁺ counterion. In some embodiments, concerning the process for preparing a dexlansoprazole composition, the organic solvent is selected from the group consisting of acetone, ethyl acetate, ethyl alcohol and mixtures thereof. In some embodiments, concerning the process for preparing a dexlansoprazole composition, the sugar alcohol is mannitol. In some embodiments, the first excipient is hydroxypropylcellulose. In some embodiments, concerning the process for preparing a dexlansoprazole composition, the second excipient is hydroxypropylcellulose. In some embodiments, concerning the process for preparing a dexlansoprazole composition, the base is Ca(OH)₂, the sugar alcohol is mannitol, the first excipient is hydroxypropylcellulose, and the organic solvent is acetone. In some embodiments, concerning the process for preparing a dexlansoprazole composition, the second mixture comprises low substitution hydroxypropylcellulose and sucrose spheres. In some embodiments, concerning the process for preparing a dexlansoprazole composition, the base is mixed with the organic solvent prior to addition of the sugar alcohol. In some embodiments, concerning the process for preparing a dexlansoprazole composition, the base mixed with the organic solvent prior to addition of the excipient. In some embodiments, concerning the process for preparing a dexlansoprazole composition, the drying includes spray drying, and drying under reduced pressure, wherein the drying occurs at, below or above room temperature.

Some embodiments provide a dexlansoprazole formulation comprising: the composition formed by a process a disclosed herein and, in addition, a pharmaceutically acceptable excipient.

In some embodiments, concerning the dexlansoprazole formulation, the base is Ca(OH)₂, the sugar alcohol is mannitol, the first excipient is hydroxypropylcellulose, and the second mixture is a mixture of low substitution hydroxypropylcellulose and sucrose spheres. In some embodiments, concerning the dexlansoprazole formulation, the dexlansoprazole is in the form of a salt or hydrate of dexlansoprazole.

Some embodiments provide a dexlansoprazole formulation comprising: dexlansoprazole, a base, and a sugar alcohol, wherein the base is selected from the group consisting of Ca(OH)₂, CaO, a mixture of CaCO₃ and NaOH, and mixtures thereof. In some embodiments, the base is Ca(OH)₂. In some embodiments, concerning the dexlansoprazole formulation, the base does not include a component selected from the group consisting of MgO and MgCO₃. In some embodiments, concerning the dexlansoprazole formulation, the base does not include a Mg²⁺ counterion. In some embodiments, concerning the dexlansoprazole formulation, the base includes a Ca²⁺ counterion. In some embodiments, concerning the dexlansoprazole formulation, the sugar alcohol is mannitol. In some embodiments, concerning the dexlansoprazole formulation, the base is Ca(OH)₂, the sugar alcohol is mannitol, and the formulation further comprises low substitution hydroxypropylcellulose, and sucrose spheres. In some embodiments, concerning the dexlansoprazole formulation, the dexlansoprazole is in the form of a salt or hydrate.

Some embodiments provide a process for preparing a dexlansoprazole composition comprising: preparing a mixture by mixing dexlansoprazole and Ca(OH)₂ in an organic solvent; and drying to provide a dexlansoprazole composition. In some embodiments, the process for preparing a dexlansoprazole composition further comprises: layering the mixture on a support matrix to provide a coated excipient mixture, wherein the drying comprises drying the coated excipient mixture to provide a dexlansoprazole composition. In some embodiments, concerning the process for preparing a dexlansoprazole composition, the organic solvent is acetone.

Some embodiments provide a dexlansoprazole formulation comprising: a dexlansoprazole composition as disclosed herein, and an additional pharmaceutically acceptable excipient.

Some embodiments provide a dexlansoprazole formulation comprising: dexlansoprazole, and Ca(OH)₂. In some embodiments, concerning the dexlansoprazole formulation comprising: dexlansoprazole, and Ca(OH)₂, the formulation further comprises one or more excipients.

Some embodiments provide a method of treating or preventing a digestive disorder in a mammal thereof comprising administering to said mammal an effective amount of a formulation as disclosed herein.

### BRIEF DESCRIPTION OF THE DRAWINGS

FIG. 1 is an XRD pattern of KAPIDEX™ formulation.

FIG. 2 is an XRD pattern of KAPIDEX™ formulation after water treatment (contains peaks from dexlansoprazole, titanium dioxide and talc).

FIG. 3 is an XRD pattern of L-HPC spheres.

FIG. 4 is an XRD pattern of HPC (KLUCEL® EF).

FIG. 5 is an XRD pattern of sucrose spheres.

FIG. 6 is an XRD pattern of calcium hydroxide.

FIG. 7 is an XRD pattern of titanium dioxide.

FIG. 8 is an XRD pattern of talc.

FIG. 9 is an XRD pattern of mannitol.

### DETAILED DESCRIPTION OF THE PREFERRED EMBODIMENT

As used herein, the term “base” refers to any base, preferably an inorganic base. For example, the base can be ammonium carbonate, ammonium hydroxide, barium carbonate, barium hydroxide, barium phosphate, calcium carbonate, calcium phosphate, calcium hydroxide, cesium carbonate, cesium hydroxide, lithium carbonate, lithium hydroxide, magnesium carbonate, magnesium phosphate, magnesium hydroxide, potassium carbonate, potassium bicarbonate, potassium hydroxide, potassium phosphate, soda lime, sodium carbonate, sodium bicarbonate, sodium hydroxide, sodium phosphate, and the like. In a typical embodiment the base can be Ca(OH)₂, CaO, CaCO₃, or mixtures thereof. In certain embodiments, the base is calcium hydroxide. In a preferred embodiment, the base is micronized. In certain embodiments, the base is neither MgO nor MgCO₃. In some embodiments, the base does not include a Mg²⁺ counterion. In some embodiments, the base includes a Ca²⁺ counterion.

As used herein, the term “sugar spheres,” is synonymous with the terms “neutral pellets,” “nonpareil seeds,” “microgranules” and “sugar beads” and refers to a combination of a natural sugar and a starch. For example, the sugar sphere can be a mixture of sucrose and corn starch.

As used herein, the term “sugar alcohol” refers to any polyol having the general formula H[HC(OH)]ₙ₊₁H. For example, the sugar alcohol can be glycol, glycerol, erythritol, threitol, arabitol, xylitol, ribitol, mannitol, sorbitol, dulcitol, iditol, and the like. In a typical embodiment, the sugar alcohol can be mannitol. In a preferred embodiment, the mannitol can be micronized.

As used herein, the term “excipient” refers to any inert ingredient, not itself a therapeutic agent, added to a pharmaceutical composition to improve its handling or storage properties or to permit or facilitate formation of a dose unit of the composition into a discrete article such as a capsule or tablet suitable for oral administration.

As used herein, the term “organic solvent” refers to any carbon containing solvent. For example, the organic solvent can be acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethyl ether, diethylene glycol, diglyme (diethylene glycol dimethyl ether), 1,2-dimethoxyethane (glyme, DME), dimethylether, dimethylformamide (DMF), dimethyl sulfoxide (DMSO), dioxane, ethanol, ethyl acetate, ethylene glycol, glycerin, heptane, hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, supercritical carbon dioxide, tetrahydrofuran (THF), toluene, triethyl amine, o-xylene, m-xylene, p-xylene, combinations thereof and the like. In a preferred embodiment, the organic solvent can be acetone.

### Methods of Preparation

Some embodiments relate to a process for preparing a dexlansoprazole composition comprising the steps of, preparing an organic mixture, for example mixing dexlansoprazole, a base, a sugar alcohol, and an excipient in a solvent, layering the organic mixture on an excipient mixture, including spraying the organic mixture onto the excipient mixture to provide a coated excipient mixture, and drying the coated excipient mixture to provide the dexlansoprazole composition. Such steps may be combined with other steps. In some embodiments, relating to the process for preparing a dexlansoprazole composition, the base can be calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment, relating to the process for preparing a dexlansoprazole composition, the base can be Ca(OH)₂, CaO, CaCO₃, or mixtures thereof. In a preferred embodiment, relating to the process for preparing a dexlansoprazole composition, the base can be Ca(OH)₂. In a preferred embodiment, relating to the process for preparing a dexlansoprazole composition, the base can be MgCO₃. In certain embodiments of the process for preparing a dexlansoprazole composition, the base does not include a component selected from the group consisting of MgO and MgCO₃. In certain embodiments of the process for preparing a dexlansoprazole composition, the base is not MgO. In certain embodiments of the process for preparing a dexlansoprazole composition, the base is not MgCO₃. In certain embodiments of the process for preparing a dexlansoprazole composition, the base does not include a Mg²⁺ counterion. In certain embodiments, the base includes a Ca²⁺ counterion. In some embodiments, relating to the process for preparing a dexlansoprazole composition, the solvent is an organic solvent selected from the group consisting of acetone, acetonitrile, 1-butanol, 2-butanol, 2-butanone, t-butyl alcohol, 1-propanol, 2-propanol, methanol, dichloromethane, diethyl ether, diethylene glycol, diglyme (diethylene glycol dimethyl ether), 1,2-dimethoxyethane (glyme, DME), dimethylether, dimethylformamide (DMF), dioxane, ethyl alcohol, ethyl acetate, ethylene glycol, glycerin, methyl t-butyl ether (MTBE), supercritical carbon dioxide, tetrahydrofuran (THF), toluene, o-xylene, m-xylene, p-xylene, combinations thereof and the like. In a typical embodiment, relating to the process for preparing a dexlansoprazole composition, the organic solvent can be acetonitrile, dichloromethane, dimethylformamide, ethyl acetate, acetone, ethyl alcohol, methanol, or mixtures thereof. In a more typical embodiment, relating to the process for preparing a dexlansoprazole composition, the organic solvent can be ethyl acetate, acetone, ethyl alcohol or mixtures thereof. In a preferred embodiment, relating to the process for preparing a dexlansoprazole composition, the organic solvent is acetone. In some embodiments, relating to the process for preparing a dexlansoprazole composition, the solvent is an aqueous solvent. In some preferred embodiments, relating to the process for preparing a dexlansoprazole composition, the base can be Ca(OH)₂, the sugar alcohol can be mannitol, the excipient can be hydroxypropylcellulose, and the organic solvent can be acetone. In certain embodiments, relating to the process for preparing a dexlansoprazole composition, the excipient mixture can be a mixture of low substitution hydroxypropylcellulose and sucrose spheres. In certain embodiments, relating to the process for preparing a dexlansoprazole composition, the drying can be performed by spray drying, drying under reduced pressure, drying at room temperature, drying below room temperature, drying above room temperature, or drying under a combination of these conditions.

Some embodiments relate to a process for preparing a dexlansoprazole composition comprising the steps of, preparing a first mixture, for example by mixing dexlansoprazole, a base, a sugar alcohol, and an excipient in a solvent, and drying to provide a dexlansoprazole composition. Such steps may be combined with other steps. Some embodiments, relating to the process for preparing a dexlansoprazole composition, further comprise layering the first mixture on a support matrix, by spraying the first mixture onto the support matrix to provide a coated excipient mixture, wherein the drying comprises drying the coated excipient mixture to provide a dexlansoprazole composition. Some embodiments, relating to the process for preparing a dexlansoprazole composition, further comprise preparing a second mixture by mixing a second excipient and support matrix; and layering the first mixture on the second mixture, by spraying the first mixture onto the second mixture to provide a coated excipient mixture, wherein the drying comprises drying the coated excipient mixture to provide a dexlansoprazole composition. In some embodiments, the base can be calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment, relating to the process for preparing a dexlansoprazole composition, the base can be Ca(OH)₂, CaO, CaCO₃, or mixtures thereof. In a preferred embodiment, relating to the process for preparing a dexlansoprazole composition, the base can be Ca(OH)₂. In a preferred embodiment, relating to the process for preparing a dexlansoprazole composition, the base can be MgCO₃. In certain embodiments of the process for preparing a dexlansoprazole composition, the base does not include a component selected from the group consisting of MgO and MgCO₃. In certain embodiments of the process for preparing a dexlansoprazole composition, the base is not MgO. In a certain embodiments of the process for preparing a dexlansoprazole composition, the base is not MgCO₃. In certain embodiments of the process for preparing a dexlansoprazole composition, the base does not include a Mg²⁺ counterion. In certain embodiments, the base includes a Ca²⁺ counterion. In some embodiments, relating to the process for preparing a dexlansoprazole composition, the solvent is an organic solvent selected from the group consisting of acetone, acetonitrile, 1-butanol, 2-butanol, 2-butanone, t-butyl alcohol, 1-propanol, 2-propanol, methanol, dichloromethane, diethyl ether, diethylene glycol, diglyme (diethylene glycol dimethyl ether), 1,2-dimethoxyethane (glyme, DME), dimethylether, dimethylformamide (DMF), dioxane, ethyl alcohol, ethyl acetate, ethylene glycol, glycerin, methyl t-butyl ether (MTBE), supercritical carbon dioxide, tetrahydrofuran (THF), toluene, o-xylene, m-xylene, p-xylene, combinations thereof and the like. In a typical embodiment, relating to the process for preparing a dexlansoprazole composition, the organic solvent can be acetonitrile, dichloromethane, dimethylformamide, ethyl acetate, acetone, ethyl alcohol, methanol, or mixtures thereof. In a more typical embodiment, relating to the process for preparing a dexlansoprazole composition, the organic solvent can be ethyl acetate, acetone, ethyl alcohol or mixtures thereof. In a preferred embodiment, relating to the process for preparing a dexlansoprazole composition, the organic solvent is acetone. In some embodiments, relating to the process for preparing a dexlansoprazole composition, the solvent is an aqueous solvent. In some preferred embodiments, relating to the process for preparing a dexlansoprazole composition, the base can be Ca(OH)₂, the sugar alcohol can be mannitol, the excipient can be hydroxypropylcellulose, and the organic solvent can be acetone. In certain embodiments, relating to the process for preparing a dexlansoprazole composition, the support matrix can be sucrose spheres. In some embodiments, the sucrose spheres can be 35-40 mesh (425-500 microns), 30-35 mesh (500-600 microns), 25-30 mesh (600-725 microns), 20-25 mesh (710-850 microns), 18-20 mesh (850-1000 microns), 16-20 mesh (850-1180 microns) and 14-18 mesh (1000-1400 microns). In a typical embodiment, the sucrose spheres can be 30-35 mesh (500-600 microns). In certain embodiments, relating to the process for preparing a dexlansoprazole composition, the drying can be performed by spray drying, drying under reduced pressure, drying at room temperature, drying below room temperature, drying above room temperature, or drying under a combination of these conditions.

Regarding the method of preparing the organic mixture, it will be appreciated by those of skill in the art that the order of addition of the components can be varied according to various preparation parameters. Additionally, it will be appreciated that some components of the mixture will be partially or fully dissolved depending on weight:volume ratio of each component and the solvent and other parameters. For example, rate of mixing and temperature may affect the solubility of a component. In some embodiments, the temperature of the solvent can range up to the boiling point of the solvent. Precautions to vary and control the temperature are appropriate under such conditions. Additionally, the initial particulate or morphic form of the component can also affect the rate of dissolution. For example, the excipient(s) can be micronized to facilitate dissolution and/or adhesion during layering process.

Some embodiments relate to a dry mix of dexlansoprazole and a base. In some embodiments the dry mix of dexlansoprazole and the base is prepared by a process comprising mixing dexlansoprazole and the base. In some embodiments, the process comprises mixing dexlansoprazole and base in a solvent; and drying to provide the dry mix of dexlansoprazole and the base. In some embodiments the base is selected from the group consisting of calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment the base can be Ca(OH)₂, CaO, CaCO₃, or mixtures thereof. In some embodiments, the base is calcium hydroxide (Ca(OH)₂). In some embodiments, the base is MgCO₃. In certain embodiments, the base does not include a component selected from the group consisting of MgO and MgCO₃. In a certain embodiments, the base is not MgO. In certain embodiments, the base is not MgCO₃. In certain embodiments, the base does not include a Mg²⁺ counterion. In certain embodiments, the base includes a Ca²⁺ counterion. In some embodiments the solvent is an organic solvent selected from the group consisting of acetone, acetonitrile, 1-butanol, 2-butanol, 2-butanone, t-butyl alcohol, 1-propanol, 2-propanol, methanol, dichloromethane, diethyl ether, diethylene glycol, diglyme (diethylene glycol dimethyl ether), 1,2-dimethoxyethane (glyme, DME), dimethylether, dimethylformamide (DMF), dioxane, ethyl alcohol, ethyl acetate, ethylene glycol, glycerin, methyl t-butyl ether (MTBE), supercritical carbon dioxide, tetrahydrofuran (THF), toluene, o-xylene, m-xylene, p-xylene, combinations thereof and the like. In a typical embodiment the organic solvent can be acetonitrile, dichloromethane, dimethylformamide, ethyl acetate, acetone, ethyl alcohol, methanol, or mixtures thereof. In a more typical embodiment, the organic solvent can be ethyl acetate, acetone, ethyl alcohol or mixtures thereof. In a preferred embodiment, the organic solvent is acetone. In some embodiments, the solvent is an aqueous solvent.

In some embodiments the weight:volume ratio of each component to solvent can be in the range of from about 0.01 g to 100 g per liter, in the range of from about 1 g to about 75 g per liter, in the range of from about 10 g to about 50 g per liter. In some embodiments the weight:volume ratio of total component to solvent can be in the range of from about 0.4 g to about 400 g per liter, in the range of from about 4 g to about 300 g per liter, in the range of from about 40 g to about 200 g per liter. In a typical embodiment, the weight:volume ratio of each component to solvent can be in the range of from about 0.01 g to 20 g per liter. In some embodiments, the solvent can be an organic solvent. For example, the solvent can be acetone. In some embodiments, the solvent can be water or a mixture of water and an organic solvent. For example the solvent can be water or a mixture of water and isopropyl alcohol. The weight:volume ratio can be adjusted depending on the solvent and the individual components of the composition.

In some embodiments, the base is suspended in a solvent prior to addition of the other components of the mixture. For example, the base can be suspended in a solvent prior to addition of dexlansoprazole. In some embodiments, the base will have greater than 90% purity, greater than 94% purity, greater than 98% purity, or greater than 99% purity. In some embodiments the base is selected from the group consisting of calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment, the base can be calcium hydroxide, calcium oxide, or a mixture of calcium carbonate and sodium hydroxide. In some embodiments, the base is calcium hydroxide (Ca(OH)₂). In some embodiments, the base is MgCO₃. In some embodiments, the base does not include a component selected from the group consisting of MgO and MgCO₃. In some embodiments, the base is not MgO. In some embodiments, the base is not MgCO₃. In certain embodiments, the base does not include a Mg²⁺ counterion. In certain embodiments, the base includes a Ca²⁺ counterion. In some embodiments, the base can be two or more components selected from the group consisting of calcium hydroxide, calcium oxide, and sodium hydroxide.

In a preferred embodiment, the base can be calcium hydroxide. In some embodiments, the calcium hydroxide can have greater than 94% purity. For example, the calcium hydroxide can be 95% pure or greater. Typical impurities of inorganic hydroxdes, such as calcium hydroxide, include inorganic carbonates, such as calcium carbonate. In some embodiments, the calcium hydroxide can be in the form of pellets, flakes, granules, powders or crystals and the like. In some embodiments, the calcium hydroxide can be micronized. In some embodiments, the weight:volume ratio of calcium hydroxide to solvent can be in the range of from about 0.4 g to about 400 g per liter, in the range of from about 4 g to about 300 g per liter, in the range of from about 40 g to about 200 g per liter. In a typical embodiment, the weight:volume ratio of each component to solvent can be in the range of from about 0.1 g to 5 g per liter. In some embodiments, the solvent can be an organic solvent. For example, the solvent can be acetone. In some embodiments, the solvent can be water or a mixture of water and an organic solvent. For example the solvent can be water or a mixture of water and isopropyl alcohol.

In some embodiments, the dexlansoprazole is mixed with a solvent containing a base prior to addition of the other components. In some embodiments, the solvent is an organic solvent. For example, the dexlansoprazole can be mixed with an organic solvent, such as acetone, containing a base such as calcium hydroxide, prior to addition of the other components. In some embodiments, the dexlansoprazole will have greater than 90% purity, greater than 94% purity, greater than 98% purity, or greater than 99% purity. In some embodiments, the dexlansoprazole can be a hydrate. In some embodiments, dexlansoprazole can exist as a particular morphic form or a mixture of morphic forms. In some embodiments, the dexlansoprazole can be in the form of pellets, flakes, granules, powders or crystals and the like. In some embodiments, the dexlansoprazole can be micronized. In some embodiments, the weight:volume ratio of dexlansoprazole to solvent can be in the range of from about 0.4 g to about 400 g per liter, in the range of from about 4 g to about 300 g per liter, in the range of from about 40 g to about 200 g per liter. In a typical embodiment, the weight:volume ratio of dexlansoprazole to solvent can be in the range of from about 1 g to 15 g per liter. In some embodiments, the solvent can be water or a mixture of water and an organic solvent. For example the solvent can be water or a mixture of water and isopropyl alcohol.

In some embodiments, the solvent is an organic solvent, and is, preferably, acetone. In some embodiments, the acetone will have 97% purity or greater, 98% purity or greater, 99% purity or greater, or 99.5% purity or greater. In some embodiments, the acetone can have greater than 99% purity. For example, the acetone can be 99.5% pure or greater. Due to its hydrophilic nature, acetone may include water as an impurity.

In some embodiments the organic solvent is selected from the group consisting of acetone, acetonitrile, 1-butanol, 2-butanol, 2-butanone, t-butyl alcohol, 1-propanol, 2-propanol, methanol, diethyl ether, diethylene glycol, diglyme (diethylene glycol dimethyl ether), 1,2-dimethoxyethane (glyme, DME), dimethylether, dimethylformamide (DMF), dioxane, ethyl alcohol, ethyl acetate, ethylene glycol, glycerin, methyl t-butyl ether (MTBE), supercritical carbon dioxide, tetrahydrofuran (THF), toluene, o-xylene, m-xylene, p-xylene, combinations thereof and the like. In a typical embodiment the organic solvent is ethyl acetate, acetone, ethyl alcohol or mixtures thereof. In a preferred embodiment, the organic solvent is acetone.

In some embodiments, the sugar alcohol is dissolved in the solvent prior to addition of the other components. In some embodiments, the base is suspended in a solvent prior to addition of the sugar alcohol. In some embodiments, the solvent is an organic solvent. For example, the base can be suspended in an organic solvent prior to addition of mannitol. In a preferred embodiment, the organic solvent is acetone. In some embodiments, the sugar has at least 98% purity, greater than 98% purity, greater than 99% purity, greater than 99.5% purity or greater than 99.9% purity. In a typical embodiment, the sugar alcohol is selected from the group consisting of glycol, glycerol, erythritol, threitol, arabitol, xylitol, ribitol, mannitol, sorbitol, dulcitol, iditol, and the like. In a preferred embodiment, the sugar alcohol can be D-mannitol. In some embodiments, the D-mannitol has greater than 99% purity. For example, the D-mannitol can be 99.9% pure or greater. In some embodiments, the weight:volume ratio of D-mannitol to organic solvent can be in the range of from about 0.4 g to about 400 g per liter, in the range of from about 4 g to about 300 g per liter, in the range of from about 40 g to about 200 g per liter. In a typical embodiment, the weight:volume ratio of sugar alcohol to solvent can be in the range of from about 5 g to 25 g per liter. In some embodiments, the solvent can be water or a mixture of water and an organic solvent. For example the solvent can be water or a mixture of water and isopropyl alcohol. In some embodiments, the D-mannitol can be dissolved in a portion of solvent and then added to in one or more portions to the mixture containing one or more components.

In some embodiments, the one of more excipients can be mixed with in the solvent prior to addition of the other components of the mixture. In some embodiments, the base is suspended in an solvent prior to addition of the one of more excipients. In some embodiments, the solvent is an organic solvent. For example, the base can be suspended in an organic solvent, such as acetone, prior to addition of hydroxypropylcellulose. In some embodiments, each excipient can have greater than 90% purity, greater than 94% purity, greater than 98% purity, or greater than 99% purity. In a typical embodiment, the one or more excipients can be selected from the group consisting of carboxymethyl cellulose, methylcellulose, hydroxypropylcellulose, low substitution hydroxypropylcellulose, TiO₂, and talc. In a preferred embodiment, the one or more excipients are selected from the group consisting of hydroxypropylcellulose, low substitution hydroxypropylcellulose, hypromellose, TiO₂, and talc. In some embodiments, the hydroxypropylcellulose can have 95% purity or greater, the hydroxypropylcellulose can have 98% purity or greater, greater than 99% purity, greater than 99.5% purity or greater than 99.9% purity. For example, the hydroxypropylcellulose can be 95% pure or greater. In some embodiments, the hydroxypropylcellulose can be in the form of pellets, flakes, granules, powders or crystals and the like. In a typical embodiment, the hydroxypropylcellulose can be in the form of a powder. In some embodiments, the weight:volume ratio of hydroxypropylcellulose to organic solvent can be in the range of from about 0.4 g to about 400 g per Liter, in the range of from about 4 g to about 300 g per liter, in the range of from about 40 g to about 200 g per liter. In a typical embodiment, the weight:volume ratio of sugar alcohol to solvent can be in the range of from about 1 g to 15 g per liter. In some embodiments, the solvent can be water or a mixture of water and an organic solvent. For example the solvent can be water or a mixture of water and isopropyl alcohol.

In some embodiments, the excipient mixture includes low substitution hydroxypropylcellulose and sucrose spheres. In some embodiments, the weight:weight ratio of low substitution hydroxypropylcellulose to sucrose spheres can be in the range of from about 1:400 to about 400:1, in the range of from about 1:40 to about 40:1, or in the range of from about 1:4 to about 4:1. In a typical embodiment, the weight:weight ratio of low substitution hydroxypropylcellulose to sucrose spheres can be in the range of from about 1:1 to about 4:1. In some embodiments, the low substitution hydroxypropylcellulose can have 95% purity or greater, the low substitution hydroxypropylcellulose can have 98% purity or greater, greater than 99% purity, greater than 99.5% purity or greater than 99.9% purity. For example, the low substitution hydroxypropylcellulose can be 95% pure or greater. In some embodiments, the low substitution hydroxypropylcellulose can be in the form of pellets, flakes, granules, powders or crystals and the like. In certain embodiments, the low substitution hydroxypropylcellulose can be in the form of a powder. In some embodiments, the sucrose spheres can have 95% purity or greater, the sucrose spheres can have 98% purity or greater, greater than 99% purity, greater than 99.5% purity or greater than 99.9% purity. For example, the sucrose spheres can be 95% pure or greater. In some embodiments, the sucrose spheres can be in the form of spheres. In some embodiments, the sucrose spheres can be 35-40 mesh (425-500 microns), 30-35 mesh (500-600 microns), 25-30 mesh (600-725 microns), 20-25 mesh (710-850 microns), 18-20 mesh (850-1000 microns), 16-20 mesh (850-1180 microns) and 14-18 mesh (1000-1400 microns). In a typical embodiment, the sucrose spheres can be 30-35 mesh (500-600 microns).

In some embodiments, the method of spraying includes fluid bed coating using top spray, bottom spray (including Wurster column) or rotor/rotary processor. The process can also be wet granulation (including high shear) and extrusion/spheronization. In some embodiments, the ratio of organic mixture to excipient mixture can be in the range of from about 1:400 to about 400:1, in the range of from about 1:40 to about 40:1, 1:4 to about 4:1.

### Formulations

Some embodiments relate to a dexlansoprazole formulation comprising, a composition prepared by any of the preceding processes. In some embodiments, relating to the dexlansoprazole formulation, the base can be calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment, relating to the dexlansoprazole formulation, the base can be Ca(OH)₂, CaO, CaCO₃, or mixtures thereof. In some embodiments, relating to the dexlansoprazole formulation, the base is calcium hydroxide (Ca(OH)₂). In some embodiments, relating to the dexlansoprazole formulation, the base is MgCO₃. In some embodiments, particularly relating to the dexlansoprazole formulation, the base does not include a component selected from the group consisting of MgO and MgCO₃. In some embodiments, particularly relating to the dexlansoprazole formulation, the base is not MgO. In some embodiments, the base is not MgCO₃. In certain embodiments, the base does not include a Mg²⁺ counterion. In certain embodiments, the base includes a Ca²⁺ counterion. In some embodiments, relating to the dexlansoprazole formulation, the solvent is an organic solvent selected from the group consisting of acetone, acetonitrile, 1-butanol, 2-butanol, 2-butanone, t-butyl alcohol, 1-propanol, 2-propanol, methanol, dichloromethane, diethyl ether, diethylene glycol, diglyme (diethylene glycol dimethyl ether), 1,2-dimethoxyethane (glyme, DME), dimethylether, dimethylformamide (DMF), dioxane, ethyl alcohol, ethyl acetate, ethylene glycol, glycerin, methyl t-butyl ether (MTBE), supercritical carbon dioxide, tetrahydrofuran (THF), toluene, o-xylene, m-xylene, p-xylene, combinations thereof and the like. In a typical embodiment, relating to the dexlansoprazole formulation, the organic solvent can be acetonitrile, dichloromethane, dimethylformamide, ethyl acetate, acetone, ethyl alcohol, methanol, or mixtures thereof. In a more typical embodiment, relating to the dexlansoprazole formulation, the organic solvent can be ethyl acetate, acetone, ethyl alcohol or mixtures thereof. In a preferred embodiment, relating to the dexlansoprazole formulation, the organic solvent is acetone. In certain embodiments, relating to the dexlansoprazole formulation, the base is calcium hydroxide (Ca(OH)₂), the sugar alcohol is mannitol, the excipient is hydroxypropylcellulose, and the organic solvent is acetone. In some embodiments, relating to the dexlansoprazole formulation, the excipient mixture can be a mixture of low substitution hydroxypropylcellulose and sucrose spheres. In some embodiments, additional excipients can be present in the formulation.

In some embodiments the weight:weight ratio of each individual component to total weight of components can be in the range of from 1:1000 to about 1000:1, in the range of from about 1:1000 to about 1:1, or in the range of from about 1:100 to about 1:2.

In some embodiments, the ratio of dexlansoprazole to total weight of components can be in the range of from 1:1000 to about 1:2, in the range of from about 1:100 to about 1:10, or in the range of from about 1:50 to about 1:25. In a typical embodiment, the weight:weight ratio of dexlansoprazole to total weight of components can be in the range of from about 1:19 to about 1:3. In some embodiments, the dexlansoprazole will have greater than 90% purity, greater than 94% purity, greater than 98% purity, or greater than 99% purity.

In some embodiments, the ratio of base to total weight of components can be from 1:1000 to about 1:2, in the range of from about 1:100 to about 1:10, or in the range of from about 1:50 to about 1:25. In a typical embodiment, the weight:weight ratio of base to total weight of components can be in the range of from about 1:100 to about 1:10. In some embodiments, the base will have greater than 90% purity, greater than 94% purity, greater than 98% purity, or greater than 99% purity. In some embodiments, relating to the dexlansoprazole formulation, the base can be calcium phosphate, magnesium phosphate, zinc phosphate, calcium sulfate, magnesium sulfate, zinc sulfate, Ca(OH)₂, Mg(OH)₂, Zn(OH)₂, CaO, MgO, ZnO, CaCO₃, MgCO₃, a mixture of CaCO₃ and NaOH, a mixture of MgCO₃ and NaOH, a mixture of ZnCO₃ and NaOH, and mixtures thereof. In a typical embodiment, the base can be calcium hydroxide, calcium oxide, or a mixture of calcium carbonate and sodium hydroxide. In some embodiments, particularly relating to the dexlansoprazole formulation, the base does not include a component selected from the group consisting of MgO and MgCO₃. In some embodiments, particularly relating to the dexlansoprazole formulation, the base is not MgO. In some embodiments, particularly relating to the dexlansoprazole formulation, the base is not MgCO₃. In certain embodiments, particularly relating to the dexlansoprazole formulation, the base does not include a Mg²⁺ counterion. In certain embodiments, particularly relating to the dexlansoprazole formulation, the base includes a Ca²⁺ counterion. In some embodiments, the base can be a mixture of two or more components selected from the group consisting of calcium hydroxide, calcium oxide, and sodium hydroxide. In a preferred embodiment, the base can be calcium hydroxide. In some embodiments, the calcium hydroxide can have greater than 94% purity. For example, the calcium hydroxide can be 95% pure or greater. In some embodiments, the calcium hydroxide can be in the form of pellets, flakes, granules, powders or crystals and the like.

In some embodiments, the weight:weight ratio of sugar alcohol to total weight of components can be in the range of from 1:1000 to about 1:2, in the range of from about 1:100 to about 1:10, or in the range of from about 1:50 to about 1:25. In a typical embodiment, the weight:weight ratio of sugar alcohol to total weight of components can be in the range of from about 1:4 to about 2:3. In some embodiments, the sugar alcohol can have 98% purity or greater, greater than 99% purity, greater than 99.5% purity or greater than 99.9% purity. In a typical embodiment, the sugar alcohol can be glycol, glycerol, erythritol, threitol, arabitol, xylitol, ribitol, mannitol, sorbitol, dulcitol, iditol, and the like. In a preferred embodiment, the sugar alcohol can be D-mannitol. In some embodiments, the D-mannitol can have greater than 99% purity.

In some embodiments, each excipient can have greater than 90% purity, greater than 94% purity, greater than 98% purity, or greater than 99% purity. In a typical embodiment, the one or more excipients can be selected from the group consisting of magnesium carbonate, sucrose, low-substituted hydroxypropyl cellulose, titanium dioxide, hydroxypropyl cellulose, hypromellose 2910, talc, methacrylic acid copolymers, polyethylene glycol 8000, triethyl citrate, polysorbate 80, glyceryl monostearate, and colloidal silicon dioxide. In a preferred embodiment, the one or more excipients can be selected from the group consisting of hydroxypropylcellulose, low substitution hydroxypropylcellulose, titanium dioxide, and talc. In some embodiments, the hydroxypropylcellulose can have 95% purity or greater, the hydroxypropylcellulose can have 98% purity or greater, greater than 99% purity, greater than 99.5% purity or greater than 99.9% purity. For example, the hydroxypropylcellulose can be 95% pure or greater.

In some embodiments, the excipient mixture can be low substitution hydroxypropylcellulose and sucrose spheres. In some embodiments, the weight:weight ratio of low substitution hydroxypropylcellulose to sucrose spheres can be in the range of from about 1:400 to about 400:1, in the range of from about 1:40 to about 40:1, or from about 1:4 to about 4:1. In a typical embodiment, the weight:weight ratio of low substitution hydroxypropylcellulose to sucrose spheres can be in the range of from about 1:1 to about 4:1. In some embodiments, the low substitution hydroxypropylcellulose can have 95% purity or greater, the low substitution hydroxypropylcellulose can have 98% purity or greater, greater than 99% purity, greater than 99.5% purity or greater than 99.9% purity. For example, the low substitution hydroxypropylcellulose can be 95% pure or greater. In some embodiments, the low substitution hydroxypropylcellulose can be in the form of pellets, flakes, granules, powders or crystals and the like. In a typical embodiment, the low substitution hydroxypropylcellulose can be in the form of a powder. In some embodiments, the sucrose spheres can have 95% purity or greater, the sucrose spheres can have 98% purity or greater, greater than 99% purity, greater than 99.5% purity or greater than 99.9% purity. For example, the sucrose spheres can be 95% pure or greater. In some embodiments, the sucrose spheres can be in the form of spheres. In some embodiments, the sucrose spheres can be 35-40 mesh (425-500 microns), 30-35 mesh (500-600 microns), 25-30 mesh (600-725 microns), 20-25 mesh (710-850 microns), 18-20 mesh (850-1000 microns), 16-20 mesh (850-1180 microns) and 14-18 mesh (1000-1400 microns). In a typical embodiment, the sucrose spheres can be 30-35 mesh (500-600 microns).

Some embodiments relate to a dexlansoprazole formulation comprising, dexlansoprazole, a base, a sugar alcohol, an excipient and a excipient mixture, wherein the base can be Ca(OH)₂, the sugar alcohol can be mannitol, an excipient can be hydroxypropylcellulose; and the excipient mixture can be a mixture of low substitution hydroxypropylcellulose and sucrose spheres. In some embodiments, additional excipients can be present in the formulation.

In some embodiments the weight:weight ratio of each individual component to total weight of components can be in the range of from 1:1000 to about 1000:1, in the range of from about 1:1000 to about 1:1, or in the range of from about 1:100 to about 1:2.

In some embodiments, the ratio of mannitol to total weight of components can be in the range of from 1:1000 to about 1:2, in the range of from about 1:100 to about 1:10, or in the range of from about 1:50 to about 1:25. In a typical embodiment, the weight:weight ratio of mannitol to total weight of components can be in the range of from in the range of from about 1:4 to about 2:3. In some embodiments, the mannitol can have 98% purity or greater, greater than 99% purity, greater than 99.5% purity or greater than 99.9% purity. In a preferred embodiment, the mannitol can be D-mannitol. In some embodiments, the D-mannitol can have greater than 99% purity. For example, the D-mannitol can be 99.9% pure or greater.

In some embodiments, the ratio of hydroxypropylcellulose to total weight of components can be in the range of from 1:1000 to about 1:2, in the range of from about 1:100 to about 1:10, or in the range of from about 1:50 to about 1:25. In a typical embodiment, the weight:weight ratio of hydroxypropylcellulose to total weight of components can be in the range of from in the range of from about 1:14 to about 1:6. In some embodiments, hydroxypropylcellulose can have greater than 90% purity, greater than 94% purity, greater than 98% purity, or greater than 99% purity. For example, the hydroxypropylcellulose can be 95% pure or greater.

In some embodiments, the ratio of low substitution hydroxypropylcellulose and sucrose spheres to total weight of components can be in the range of from 1:1000 to about 1:2, in the range of from about 1:100 to about 1:10, or in the range of from about 1:50 to about 1:25. In some embodiments, the weight:weight ratio of low substitution hydroxypropylcellulose to sucrose spheres can be in the range of from about 1:400 to about 400:1, in the range of from about 1:40 to about 40:1, or from about 1:4 to about 4:1. In a typical embodiment, the weight:weight ratio of low substitution hydroxypropylcellulose to sucrose spheres can be in the range of from about 1:1 to about 4:1. In some embodiments, the low substitution hydroxypropylcellulose can have 95% purity or greater, the low substitution hydroxypropylcellulose can have 98% purity or greater, greater than 99% purity, greater than 99.5% purity or greater than 99.9% purity. For example, the low substitution hydroxypropylcellulose can be 95% pure or greater. In some embodiments, the low substitution hydroxypropylcellulose can be in the form of pellets, flakes, granules, powders or crystals and the like. In a typical embodiment, the low substitution hydroxypropylcellulose can be in the form of a powder. In some embodiments, the sucrose spheres can have 95% purity or greater, the sucrose spheres can have 98% purity or greater, greater than 99% purity, greater than 99.5% purity or greater than 99.9% purity. For example, the sucrose spheres can be 95% pure or greater. In some embodiments, the sucrose spheres can be in the form of spheres.

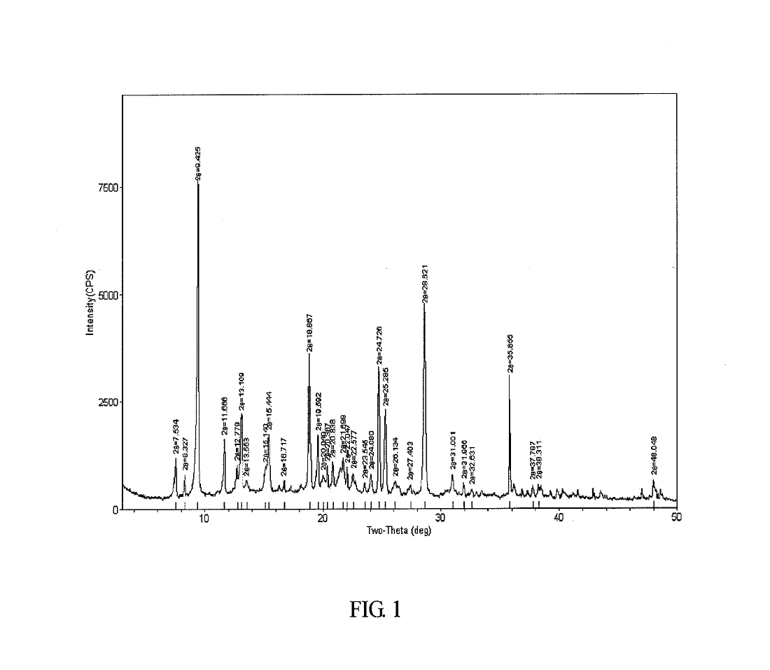

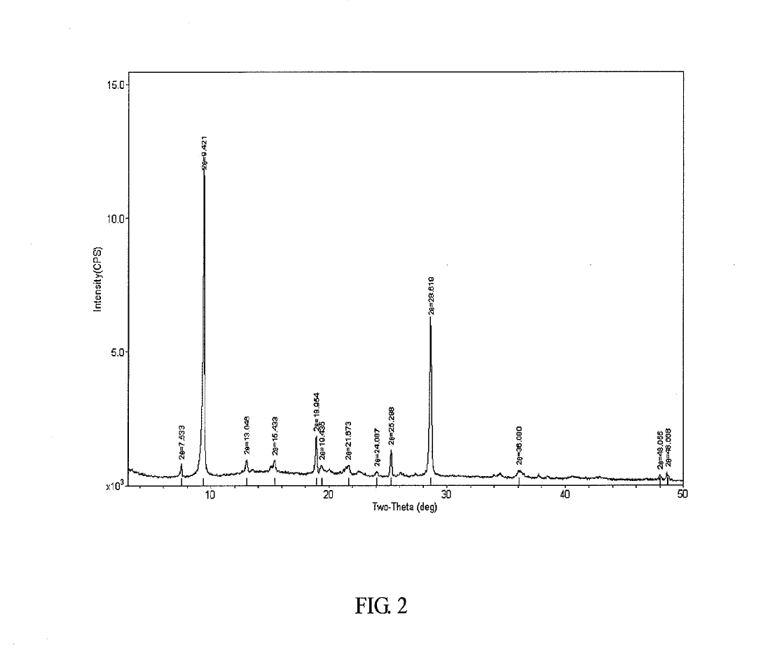

The PXRD pattern of the commercial product sold as KAPIDEX™ (FIG. 1), displays characteristic X-ray diffraction peaks relating to sucrose spheres, titanium dioxide and talc. After treatment with water to remove water soluble inert ingredients, such as sugar spheres, the water treated commercial product sold under KAPIDEX™, (FIG. 2), displays, characteristic X-ray diffraction peaks relating to titanium dioxide and talc. The peaks corresponding to sugar spheres, however, are no longer present.

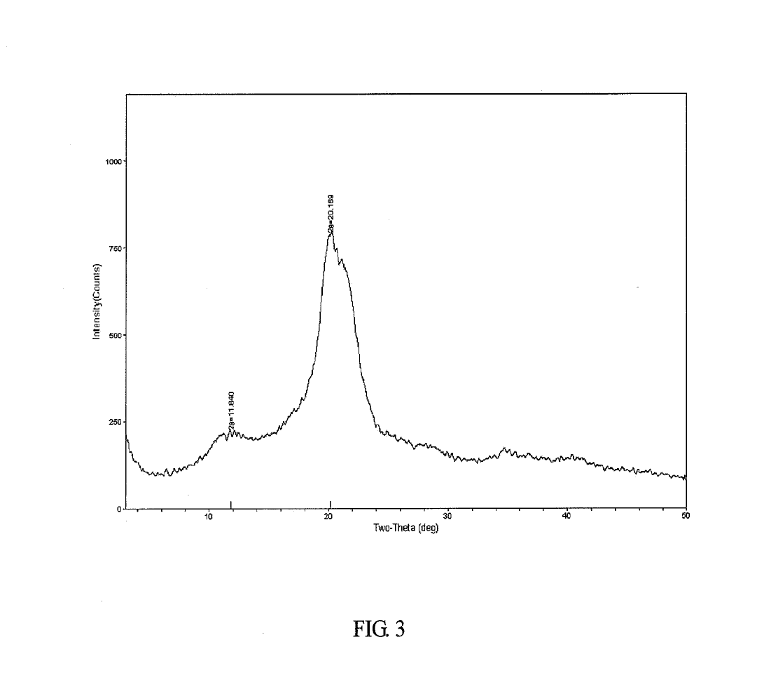

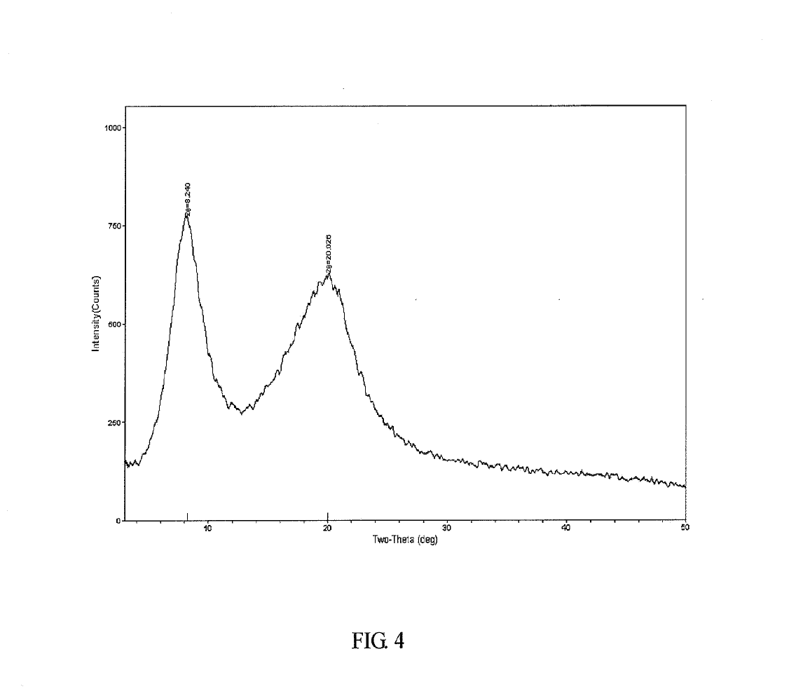

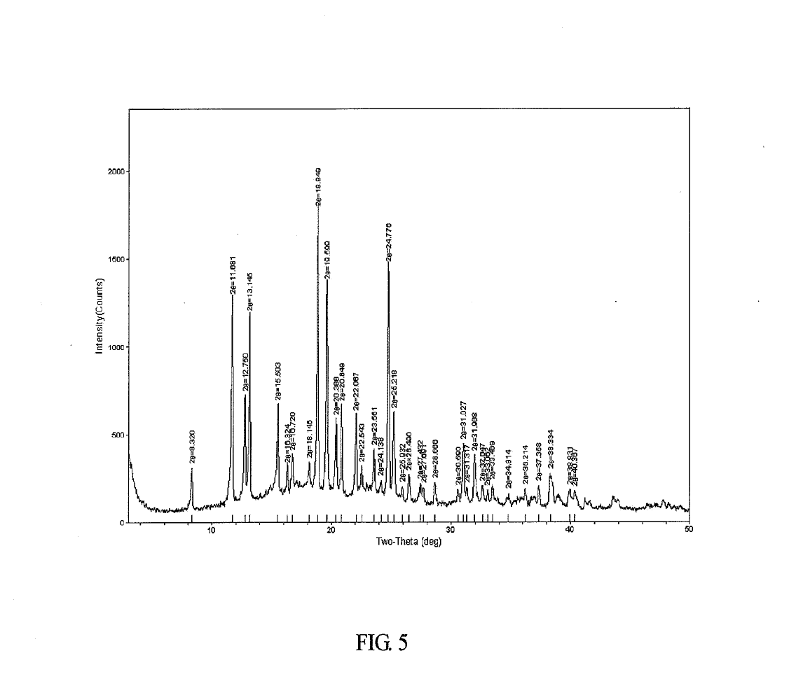

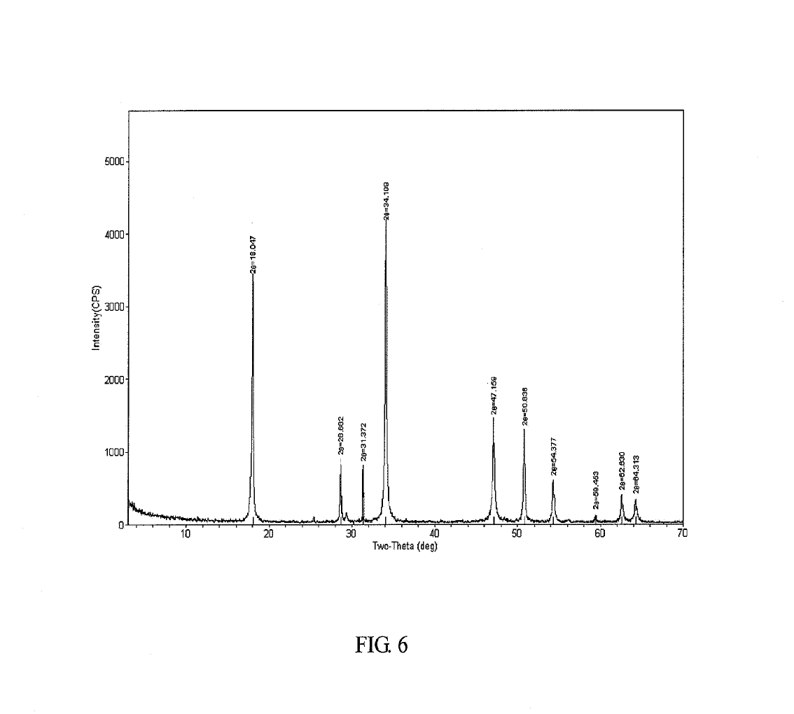

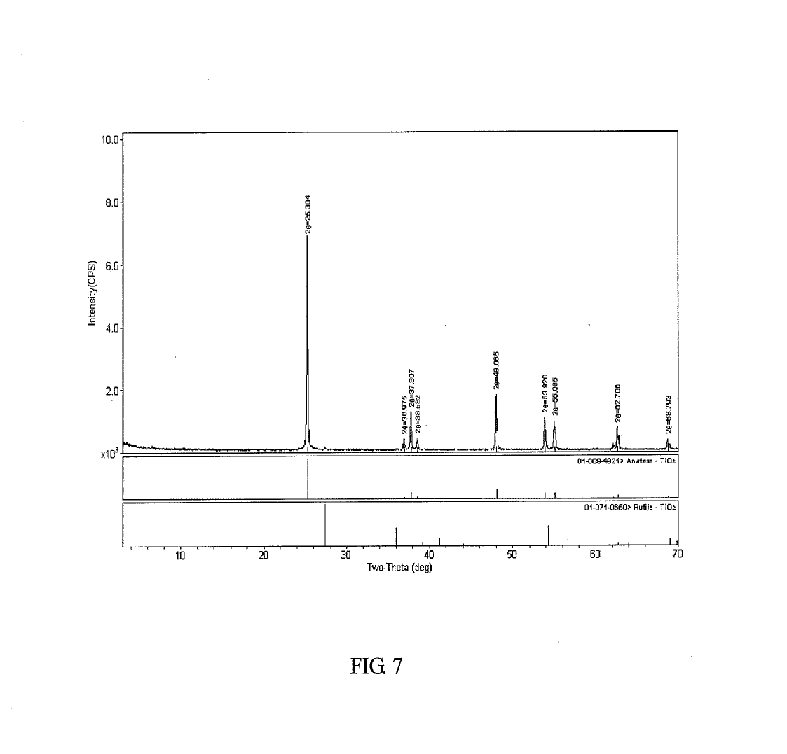

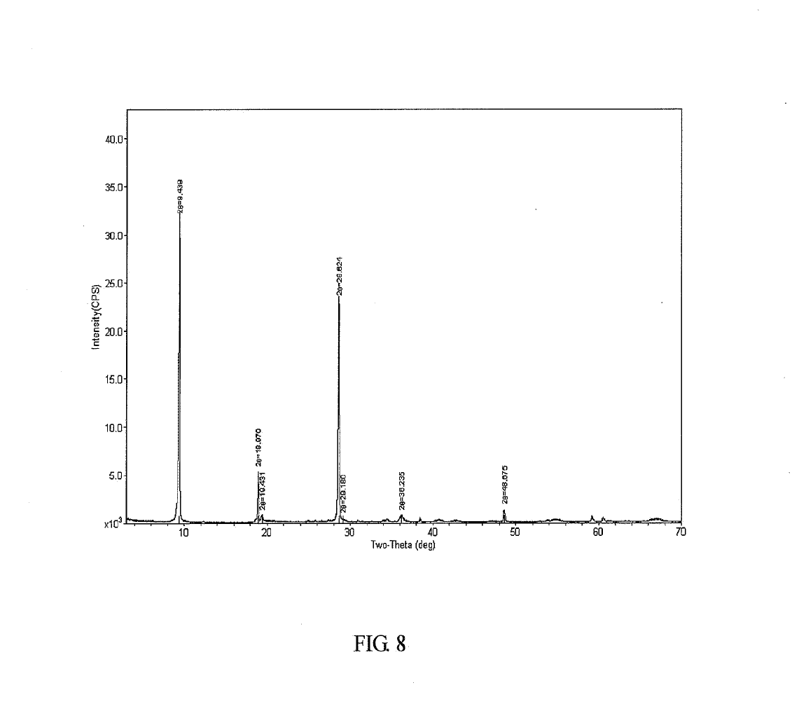

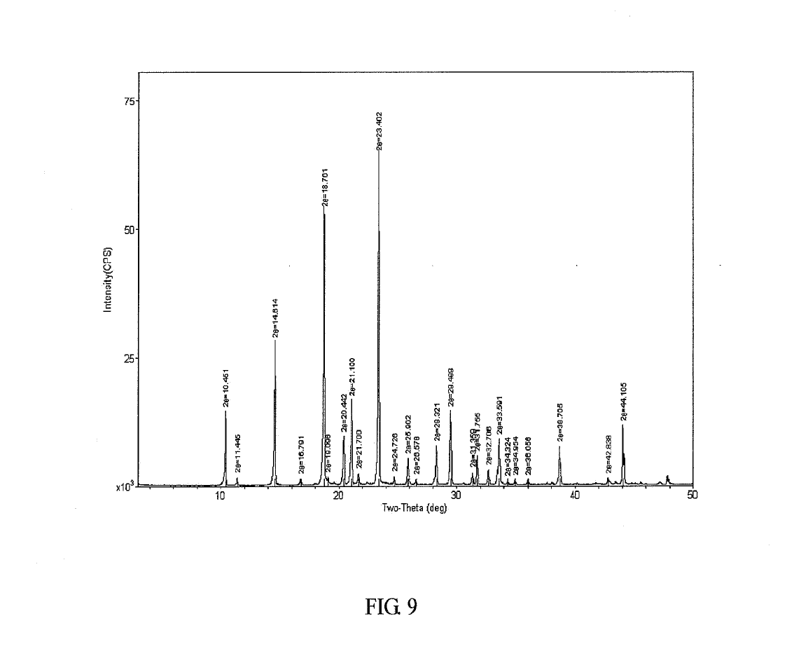

The X-ray diffraction patterns of formulations containing inactive ingredients, (i.e. excipients) requires careful analysis. The presence of peaks relating to the inactive ingredients can be obscuring and an identification of the peaks relating to the inactive ingredients can be helpful in analysis of the pattern. For analysis of the formulations containing inactive ingredients a set of X-ray diffraction pattern for certain inactive ingredients were obtained. The X-ray diffraction pattern of both L-HPC spheres (FIG. 3), and HPC (KLUCEL® EF) (FIG. 4), display very broad peaks with weak intensity. The X-ray diffraction pattern of sucrose spheres (FIG. 5), calcium hydroxide (FIG. 6), titanium dioxide (FIG. 7), talc (FIG. 8), and mannitol (FIG. 9) display relatively sharp peaks with medium to high intensity.

This sample shows significant stability at 60° C. and 60% relative humidity for a period of days and even up to two weeks. Thus, sample exhibits favorable properties for pharmaceutical formulations.

Some embodiments relate to a dexlansoprazole formulation comprising dexlansoprazole, a base, a sugar alcohol, an excipient and a excipient mixture, wherein the base can be Ca(OH)₂, the sugar alcohol can be mannitol, an excipient can be hydroxypropylcellulose; and the excipient mixture can be a mixture of low substitution hydroxypropylcellulose and sucrose spheres. In some embodiments, the formulation exhibits one or more of the most intense PXRD peaks listed in Table 1. In some embodiments, the formulation exhibits two or more PXRD peaks listed in Table 1. In some embodiments, the formulation exhibits three or more PXRD peaks listed in Table 1. In some embodiments, the formulation exhibits four or more PXRD peaks listed in Table 1. In some embodiments, the formulation exhibits five or more PXRD peaks listed in Table 1. In some embodiments, the formulation exhibits six or more PXRD peaks listed in Table 1. In some embodiments, the formulation exhibits seven or more PXRD peaks listed in Table 1.

Some embodiments provide a dexlansoprazole formulation comprising: dexlansoprazole, a base, and a sugar alcohol. In some embodiments, the formulation exhibits two or more PXRD peaks selected from Table 1. In some embodiments, the formulation exhibits three or more PXRD peaks selected from Table 1. In some embodiments, the formulation exhibits four or more PXRD peaks selected from Table 1. In some embodiments, the formulation exhibits five or more PXRD peaks selected from Table 1. In some embodiments, the formulation exhibits six or more PXRD peaks selected from Table 1. In some embodiments, the formulation exhibits seven or more PXRD peaks selected from Table 1. In some embodiments, the base is selected from the group consisting of Ca(OH)₂, CaO, a mixture of CaCO₃ and NaOH, and mixtures thereof. In some embodiments, the base is Ca(OH)_{z}. In some embodiments, the base does not include a component selected from the group consisting of MgO and MgCO₃. In some embodiments, the base is not MgO. In some embodiments, the base is not MgCO₃. In certain embodiments, the base does not include a Mg counterion. In certain embodiments, the base includes a Ca counterion.

TABLE 1
X-ray diffraction pattern

| Entry | d (Angstroms) |
|---|---|
| 1 | 17.15 |
| 2 | 5.74 |
| 3 | 4.43 |
| 4 | 3.58 |
| 5 | 3.55 |
| 6 | 2.55 |
| 7 | 2.22 |
| 8 | 1.99 |
| 9 | 1.83 |
| 10 | 1.77 |
| 11 | 1.72 |
| 12 | 1.60 |
| 13 | 1.51 |
| 14 | 1.27 |
| 15 | 1.12 |

### Encapsulation Coating

In some embodiments, the formulation includes an encapsulation coating. The encapsulation coat may include different combinations of pharmaceutical active ingredients, hydrophilic surfactant, lipophilic surfactants and triglycerides. In some embodiments, the solid pharmaceutical composition includes a solid carrier, the solid carrier being formed of different combinations of pharmaceutical active ingredients, hydrophilic surfactant, lipophilic surfactants and triglycerides.

Encapsulation, for example, may be conducted by traditional pan coating or fluidized bed techniques. Several process (air supply, temperature, spray rate, spray system, powder feed, and attrition) and formulation factors determine the quality of the end product, and one skilled in the art can readily adjust such parameters as needed.

In some embodiments, a subject formulation will include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

In some embodiments, a dexlansoprazole composition can be formulated together with one or more pharmaceutical excipients and coated with an enteric coating, as described in U.S. Pat. No. 6,346,269. For example, the dexlansoprazole composition can be uncoated or coated with an enteric coating layer. In some embodiments, one or more layers of seal coat can also be applied to the dexlansoprazole composition for protecting the active from degrading due to enteric coating. Suitable enteric-soluble coating materials, if desired, include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

### Shells

In some embodiments, the formulation can include a shell. As used herein “shell” refers to a barrier that encapsulates, surrounds, or encompasses at least a portion of a material or an object. A variety of specific materials and methods for the formation of such shells, are well known to those of ordinary skill in the art.

In some embodiments, the shell may be either a hard or soft capsule shell, and can include a number of fundamental constituents, namely a matrix forming material, and optionally at least one plasticizing agent. A wide variety of matrix forming materials are suitable for use in the dosage forms of the present embodiment, and the selection of specific materials may be based, at least in part, on factors such as the specific results to be achieved. Examples of specific materials include, but are not limited to, gelatins, including type A gelatins, such as the gelatin derived from acid-treated pigskins, and type B gelatins, such as those derived from alkali-treated bovine bones and hides, hydroxypropyl methylcellulose (HPMC), starches, and gum acacia. Other specific matrix forming materials that may be particularly desired in view of a given overall dosage form can be determined by those of ordinary skill in the art.

The specific amount of matrix forming material used in the shell formulation may be determined in part by a variety of factors, including the type of shell to be formed (i.e. hard or soft), and by the amount and type of other constituents or additives that are to be included in the shell. However, in one aspect, the amount of matrix forming material may be from about 10% w/w to about 100% w/w of the shell. In another aspect, the amount of matrix forming material may be from about 20% w/w to about 70% w/w of the shell. In another aspect, the amount may be from about 30% w/w to about 50% w/w of the shell. In one embodiment, the amount of matrix forming material can be 100% of the shell. For example, the matrix forming material can be 100% HPMC (after water is removed during processing). In another embodiment, the matrix forming material can include a gelling agent. In another embodiment, the matrix forming material can include a gelling aid/promoter. In another embodiment, the matrix forming material can include both gelling agent and gelling aid/promoter. For example, both carrageenan, a gelling agent, and potassium chloride, a gelling aid/promoter, can be included in a HPMC based formulation.

Many plasticizing agents are known, and may also be used in the shell of the present dosage form. One basis for selecting a particular plasticizing agent may be the solubility of that agent in a specific hydrophilic fill material to be used. In one aspect, the plasticizing agent may have a solubility of less than about 10% w/w in the fill material. In another aspect, the solubility of the plasticizing agent in the fill material may be less than about 5% w/w. In yet another aspect, the solubility may be less than about 1% w/w. In a further aspect, the solubility of the plasticizing agent may be less than about 0.5% w/w. Lowered solubility in the specific hydrophilic fill material substantially impedes the migration of the plasticizing agent out of the shell and into the fill material. Examples of specific plasticizing agents displaying such limited solubilities in many hydrophilic surfactant materials include, but are not limited to, sorbitol, sorbitanes, xylitol, maltitol, maltitol syrup, partially dehydrated hydrogenated glucose syrups, hydrogenated starch hydrolysate, polyhydric alcohols having an equilibrium relative humidity of greater than or equal to 80%, carrageenan, polyglycerol, non-crystallizing solutions of sorbitol, glucose, fructose, glucose syrups, and mixtures and equivalents thereof.

Whether the plasticizing agent selected and used is one that has a low solubility in the fill material or not, in accordance with one aspect of the invention, the plasticizing agent may be presented in an amount that is sufficient to maintain an effective shell plasticity upon migration of a portion of the plasticizing agent from the shell and into the fill and/or may be present in a sufficient amount to maintain a desirable dissolution/disintegration profile with respect to the rate and the extent release and/or dispersing of the encapsulated active agent in a specific dissolution medium or upon administration inside the GI tract. The exact amount of plasticizing agent required to compensate for the plasticizing agent anticipated to be lost may depend on a variety of factors, such as the specific fill material and solubility of the plasticizing agent therein. However, those of ordinary skill in the art will be able to readily determine approximate amounts required to maintain effective shell plasticity based on the known characteristics presented by a given dosage form, and will further be able to identify specific amounts through routine experimentation with the dosage form. In one aspect of the invention, such an amount of plasticizing agent may be from about 4% w/w to about 60% w/w of the shell. In another aspect, the amount may be from about 10% w/w to about 35% w/w.

An additional option for maintaining effective shell plasticity and/or a desirable dissolution/disintegration profile of the encapsulated active agent in view of the highly hydrophilic fill material is to include a combination of plasticizing agents in the shell in a total amount sufficient to maintain effective shell plasticity upon migration of a portion of either or both agents into the fill material. In one aspect of the invention, such a combination may include a first plasticizing agent, and a second plasticizing agent having a limited solubility in the fill material as recited above. The total amounts and ratios of each ingredient required to maintain an effective plasticity may be determined by one of ordinary skill in the art in the manners already indicated. While a variety of ratios and amounts are contemplated, in one aspect, the total amount of combined plasticizing agent may be within the ranges already established for plasticizing agents herein.

In addition to the components of a matrix forming material and the at least one plasticizing agent, the shells used in the dosage forms of the present embodiments may include additional additives as required, in order to achieve a specifically desired formulation or result. Examples of such additives may include, but are not limited to, coloring agents, antioxidants, preservatives, surfactants, and mixtures thereof. Specific amounts of these additives, as well as others not specifically recited will be readily determined by those of ordinary skill in the art, consistent with a working knowledge thereof, and the principles set forth herein.

In addition to the above recited devices and methods for maintaining the flexibility, or plasticity of a shell encapsulating a highly hydrophilic material, another approach encompassed by the present invention, is the use of a hydrophobic coating on a surface of the shell. Specifically, it is thought that by placing a hydrophobic coating along an inner surface of the shell, that water and plasticizer may be effectively stopped from migrating into the fill material, or at least that such migration may be slowed. Further, when such a coating is provided along an outer surface of the shell it is thought that the coating prevents the absorption of moisture from the outside environment, and its resultant migration into the fill material, or that at least, such is slowed. In addition to slowing or preventing the migration of water and plasticizers into the fill material, use of such coatings is thought to prevent or slow the migration of plasticizers from the shell and into the fill material. Such migration is known to cause over-softening or “sweating” of the shell, which can be can be as detrimental to the performance of the dosage form as embrittling of the shell.

Either coating may be used separately in various embodiments of the present invention, or a combination of coatings may be used. Such coatings may further be employed with virtually any specific dosage form or shell formulation as contemplated herein. Further, a variety of hydrophobic, or water impermeable materials may be used for the coating as will be recognized by those of ordinary skill in the art, such as oils, petroleum waxes, etc.

### Dosages

The selected dosage level can depend upon, for example, the route of administration, the severity of the condition being treated, and the condition and prior medical history of the patient being treated. It will be understood, however, that the specific dose level for any particular patient can depend upon a variety of factors including the genetic makeup, body weight, general health, diet, time and route of administration, combination with other drugs and the particular condition being treated, and its severity.

In some embodiments, the composition comprises an amount of the at least one active ingredient less than the amount of the same in a comparable composition containing an amount of the at least one active ingredient required for similar efficacy. In some embodiments, the composition comprises an amount of the at least one active ingredient more than the amount of the same in a comparable composition containing an amount of the at least one active ingredient required for similar severity and/or frequency of side effects. In some embodiments, the dosage of the at least one active ingredient may range from about 0.01 to about 1000 mg/kg. In some embodiments, the dosage may range from about 1 to about 50 mg/kg. In some embodiments, the dosage may range from about 1 to about 10 mg/kg. In some embodiments, the dosage is less than about 1,000 mg/kg, 750 mg/kg, 500 mg/kg, 300 mg/kg, about 100 mg/kg, about 50 mg/kg, about 20 mg/kg, about 10 mg/kg, about 6 mg/kg, about 3 mg/kg, about 2 mg/kg or about 1 mg/kg.

An oral composition described herein may be administered or prescribed in a dosage less than, for example, about 750 mg/kg, about 500 mg/kg, about 300 mg/kg, or about 150 mg/kg.

A composition described herein can be prescribed or administered at a constant dose, or the dosage can change as a function of treatment time. For example, dosages may increase or decrease with time in a step-wise or continuous manner. The dosage may vary depending on the effect of the dosage on a condition being treated and the occurrence of adverse side effects. For example, the patient may be instructed to continue to lower the dosage until a side effect is reduced to an acceptable level. As another example, the patient may be instructed to continue to lower the dosage until the dosage is no longer effective and then slightly increase the dosage.

In some embodiments, a composition described herein can be prescribed or administered at a specific dosage per day. In other embodiments, the patient can be instructed to take the composition when he or she experiences one or more symptoms related to a condition being treated. For example, the patient may be instructed to take a composition when he is experiencing severe pain.

### Preparation of Compositions

For oral administration, the compositions may be formulated as pills, tablets, powders, granules, dragees, capsules, liquids, sprays, gels, syrups, slurries, suspensions and the like, in bulk or unit dosage forms, for oral ingestion by a patient to be treated. The composition may be an oral dosage form, and the oral dosage form may be a solid oral dosage form. The compositions can be formulated readily, for example, by combining the active compound with any suitable pharmaceutically acceptable carrier or excipient. In a preferred embodiment, the compositions may be formulated as tablets, pills, tablets, powders, or capsules.

Pharmaceutical preparations for oral use can be obtained by mixing one or more solid excipients with a pharmaceutical composition as described herein, optionally grinding the resulting mixture, and processing the mixture of granules, after adding suitable inert ingredients, if desired, to obtain tablets, pills, tablets, powders, or capsules. Formulations of the present embodiments contain excipients. Excipients include lubricants, binders, disintegrants, preservatives, antioxidants, coloring agents, sweetening agents, souring agents, bubbling agents and flavorings.

Excipients include, for example, lactose, sucrose, D-mannitol, starch, cornstarch, crystalline cellulose, light silicic anhydride, titanium oxide, magnesium stearate, sucrose fatty acid esters, polyethylene glycol, talc, stearic acid, sodium carboxymethyl cellulose, methyl cellulose, hydroxymethyl cellulose, hydroxyethyl cellulose, hydroxypropyl cellulose, hydroxypropylmethyl cellulose, low substitution hydroxypropyl cellulose, crystalline cellulose, α-starch, gum arabic powder, gelatin, pullulan, crosslinked polyvinylpyrrolidone (povidone, PVP), sodium crosslinked carmellose, calcium carmellose, sodium carboxymethyl starch, cornstarch, crosslinked povidone (e.g. 1-ethenyl-2-pyrrolidinone homopolymer, including polyvinylpyrrolidone (PVPP) and 1-vinyl-2-pyrrolidinone homopolymer), sodium polyacrylate, polyvinyl alcohol, sodium alginate, guar gum, sodium carbonate, sodium bicarbonate, disodium hydrogenphosphate, potassium carbonate, potassium bicarbonate, heavy magnesium carbonate, magnesium carbonate, magnesium oxide, magnesium hydroxide, magnesium metasilicate aluminate, magnesium silicate, magnesium aluminate, synthetic hydrotalcite, alumina hydroxide magnesium, calcium carbonate, calcium hydroxide, polyethylene glycol, propylene glycol, benzyl benzoate, ethanol, trisaminomethane, cholesterol, triethanolamine, sodium carbonate, sodium citrate, stearyltriethanolamine, sodium lauryl sulfate, laurylaminopropionic acid, lecithin, benzalkonium chloride, benzethonium chloride, monostearic glycerol, polyvinyl alcohol, glucose, D-sorbitol, sodium chloride, glycerol, benzyl alcohol, p-oxybenzoic acid esters, chlorobutanol, benzyl alcohol, phenethyl alcohol, dehydroacetic acid, sorbic acid, sulfites, ascorbic acid, α-tocopherol, Food Color Yellow No. 5, Food Color Red No. 2, Food Color Blue No. 2, red oxide, sodium saccharin, dipotassium glycyrrhetinate, aspartame, stevia and thaumatin, citric acid (citric anhydride), tartaric acid, malic acid, and flavorings, such as lemon, lime, orange, menthol and strawberry.

In some embodiments, the excipients can be selected from the group consisting of lactose, sucrose, starch powder, maize starch or derivatives thereof, cellulose esters of alkanoic acids, cellulose alkyl esters, talc, TiO₂, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, methyl cellulose, hydroxypropylcellulose, hydroxypropylmethylcellulose, sodium carboxymethylcellulose, polyvinylpyrrolidone (PVP), and the like. In a typical embodiment, the excipients can include one or more of HPC, L-HPC, talc, TiO₂, or sugar spheres. In some embodiments, the excipient does not include a component selected from the group consisting of MgO and MgCO₃. In some embodiments, the excipient does not include a base having a Mg²⁺ counterion. In some embodiments, the excipient includes a base having a Ca²⁺ counterion.

From the foregoing, it will be obvious to those skilled in the art that various modifications in the above-described methods, and compositions can be made without departing from the spirit and scope of the invention. Accordingly, the invention may be embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein.

The present invention is more clearly understood from the following detailed description taken in conjunction with the accompanying figures. It will be understood that the examples provided herein are only for the purpose of description, not the limitation to the invention.

### EXAMPLES

### Example 1

### Formulation of Dexlansoprazole

Ca(OH)₂ (0.1 g) was combined with acetone (6 mL) with mixing, to the mixing mixture was added Dexlansoprazole (1 g), mannitol (1.17 g) and hydroxypropylcellulose (HPC) (0.18 g) with continued mixing. The mixture was applied to a clean glass plate and the solvent was allowed to evaporate to provide Sample 2 as a solid. The X-ray diffraction of the formulation exhibits a 20 peak at 6.4, 10.0, 11.0, 11.2, 12.7, 13.9, and 17.4.

### Example 2

### Formulation of Dexlansoprazole Including Layering on L-HPC and Sucrose Spheres

Ca(OH)₂ (12.6 g) was combined with acetone (360 L) with mixing, to the mixing mixture was added Dexlansoprazole (35 g), Ca(OH)₂ mannitol (17.5 g) and hydroxypropylcellulose (HPC) (3.5 g). The mixture was sieved through 80 mesh and then sprayed onto low substitution hydroxypropylcellulose (L-HPC) (31.77 g) and sucrose spheres (34.09 g) including Ca(OH)₂ (0.31 g) and hydroxypropylcellulose (HPC) (3.82 g), then dried by fluidization bed to provide a solid. The X-ray diffraction of the formulation exhibits a 20 peak at 5.2, and 6.4. Source: Cu (40 kV, 250 mA). Wavelength to compute d-spacing=1.54059 (Cu/K-alpha1).

### Example 3

Characterization of Dexlansoprazole formulation (KAPIDEX™ of Takeda Pharmaceuticals North America, Inc.), by X-Ray Diffraction

The X-ray diffraction of a dexlansoprazole formulation sold commercially under KAPIDEX™ (Takeda Pharmaceuticals North America, Inc.) was determined. The PXRD pattern of this formulation exhibits 20 peak at 7.5, 15.4, 21.7, and 24.1.

Certain inactive ingredients were removed by water treatment of the dexlansoprazole formulation sold commercially under KAPIDEX™ (Takeda Pharmaceuticals North America, Inc.). The X-ray diffraction of the formulation, after water treatment (with water soluble carbohydrates removed), exhibits 20 peaks at 7.5, 15.4, 21.7, and 24.1. Source: Cu (40 kV, 250 mA). Wavelength to compute d-spacing=1.54059 (Cu/K-alpha1).

### Example 4

### Stability Studies of Dexlansoprazole Formulations

TABLE 2

| | Sample 1 | | Sample 3¹ | Sample 4² | Sample 5³ | Sample 6⁴ |
|---|---|---|---|---|---|---|
| Time | (Uncoated Drug | | (Coated Drug | (Coated Drug | (Coated Drug | (Coated Drug |
| Point | Pellets) | Sample 2 | Pellets) | Pellets) | Pellets) | Pellets) |
| Initial | 0.219% | 0.000% | n/a | n/a | n/a | 0.23% |
| 3rd day | 0.234% | 0.177% | 0.771% | 0.657% | 0.310% | 0.24% |
| 1st week | 0.376% | 0.585% | 1.336% | 2.099% | 0.310% | 0.25% |
| 2nd week | 1.245% | 0.673% | 2.617% | 6.010% | 0.439% | 0.32%-0.34% |

¹Formulation contains dexlansoprazole in the mixture of Ca(OH)₂, mannitol (API:Ca(OH)₂:Mannitol = 1:0.4:0), HPC and acetone
²Formulation contains dexlansoprazole in the mixture of Ca(OH)₂, mannitol (API:Ca(OH)₂:Mannitol = 1:0.2:1.5), HPC and acetone
³Formulation contains dexlansoprazole in the mixture of Ca(OH)₂, mannitol (API:Ca(OH)₂:Mannitol = 1:0.2:1.5), HPC, SDS and sodium chloride and acetone
⁴Formulation contains dexlansoprazole in the mixture of Ca(OH)₂, mannitol (API:Ca(OH)₂:Mannitol = 1:0.2:1.5), HPC, SDS and acetone

The stability of samples including dexlansoprazole and Ca(OH)₂ are shown in Table 2. The materials were placed into stability chambers under the condition of 60° C. and 60% Relative Humidity (R.H.). Samples were removed from the chamber and tested for related substances after 3, 7, and 14 days. After 1 week less than 7% degradation was seen in all samples containing Ca(OH)₂. See Table 2. Additionally, the samples containing the new formulation of dexlansoprazole show less than 2% degradation after 2 weeks under test conditions.

### Preparation of Sample 3:

### Stage 1 (Drug Layering)

Micronized calcium hydroxide (9.0 g) was added into a container containing acetone (260 mL) followed by L-HPC (26 g) and MgO (15 g). The mixture was mixed for at least 60 min. Subsequently, micronized dexlansoprazole (22 g) was added and mixed for at least 30 min. Caution should be taken to avoid contact with the dispersion. The mixture was then sprayed onto sugar spheres (50 g) previously placed into a fluid-bed coater equipped with bottom spraying (inlet air temperature: 28° C.; product temperature: 27° C.; coating solution spray rate: 2.67 g/min; and spray air pressure: 1 bar) to provide dexlansoprazole coated sugar spheres (96 g). A portion of the material was retained for seal coating.

### Stage 2 (Preparing Seal Coating Mixture)

Mixture 1: A 10% HPMC mixture (g/g) was made by adding HPMC (4.2 g) to water and mixing, the mixture was retained for further processing. A talc (talc sieved through 120 mesh screen; 1.2 g) and Ca(OH)₂ (0.6 g) mixture was made by mixing talc and Ca(OH)₂ in water (40.2 g). The talc and Ca(OH)₂ mixture was blended with the retained 10% HPMC mixture.

Mixture 2: A 10% HPMC mixture (g/g) was made by adding HPMC (1.27) to water (40 g) and mixing, the mixture was retained for further processing. A talc (talc sieved through 120 mesh screen; 1.62 g) and L-HPC (4.28 g) mixture was made by mixing talc and L-HPC. The talc and L-HPC was blended with the retained 10% HPMC mixture.

### Stage 3 (Seal Coating):

Fluid bed Processor: Prewarm a fluid bed processor machine (inlet temp. 50° C. with appropriate air flow). The coated sugar spheres were added to the fluid bed processor machine fluidization (Inlet temp.: 50° C.; Product temp.: 30-38° C. (set 40° C.); atomized air: 1.0 bar; nozzle: 1.0 mm; and spray at a speed of 1.3 mL/min) was commenced using mixture 1 and mixture 2. The resulting coated material was dried at 65° C. for 1 hours in fluid bed to provide the seal coated dexlansoprazole coated sugar spheres. The material from multiple batches was retained for enteric coating.

### Stage 4 (Enteric Coating):

Eudragit L100-55 layering mixture: Eudragit L100-55 (30.39 g) was mixed with isopropyl alcohol (485 mL) in a suitable container. The Eudragit L100-55 mixture was retained for further processing. Talc (9.12 g) and TiO₂ (3.04 g) (both sieved through a 120 mesh screen), PEG6000 (6.08 g) and Polysorbate 80 (1.38 g) were added to the Eudragit L100-55 mixture with thorough further mixing.

Fluid bed Processor: Prewarm a fluid bed processor machine (inlet temp. 40° C. with appropriate air flow). The seal coated dexlansoprazole coated sugar spheres (250 g) were added to the fluid bed processor machine, fluidization (Inlet temp.: 40° C.; Product temp.: 38-40° C. (set 40° C.); atomized air: 1.8 bar; nozzle: 1.0 mm; and spray at a speed of 0.65 mL/min) was commenced using mixture 1 and mixture 2. The resulting enteric coated material was dried at 40° C. for 0.5 hours and 55° C. for 1 hour in fluid bed to provide the enteric coated dexlansoprazole coated sugar spheres. The enteric coated dexlansoprazole coated sugar spheres were tested for impurities.

### Preparation of Sample 4:

Stage 1 (Drug Layering): Micronized calcium hydroxide (24 g) was added into a container containing acetone (426.6 g) and mixed for at least 15 min, then micronized dexlansoprazole (128.6 g) was added and mixed for at least 45 additional minutes. The calcium hydroxide and dexlansoprazole mixture was retained for further processing. Hydroxypropylcellulose, NF (KLUCEL® EF PHARM; 67.4 g) was added into a container containing acetone (355.5 g) and mixed until the solid dissolved. The Hydroxypropylcellulose, NF (KLUCEL® EF PHARM) mixture was combined with the calcium hydroxide and dexlansoprazole mixture and the resulting mixture was mixed for at least 0.5 hours. The mixture was retained for further processing. Micronized mannitol (180 g) was added into a container containing acetone (696.8 g) and mixed for at least 1.5 hours. The mannitol mixture was retained for further processing. The calcium hydroxide, dexlansoprazole, Hydroxypropylcellulose, NF (KLUCEL® EF PHARM) mixture was combined with the mannitol mixture and the resulting mixture was mixed for at least 6 hours. The mixture was retained for further processing. The mixture was then sprayed onto sugar spheres (100 g) previously placed into a fluid-bed coater equipped with bottom spraying (inlet air temperature: 31-33° C.; product temperature: 31-32° C.; coating solution spray rate: 2.67 g/min; and spray air pressure: 1 bar) to provide dexlansoprazole coated sugar spheres. A portion of the material was retained for seal coating.

### Stage 2 (Preparing Seal Coating Mixture):

Seal Coating Mixture: A 10% HPMC mixture (g/g) was made by adding HPMC (2.66 g) to water and mixing, the mixture was retained for further processing. Dissolve mannitol (17.34 g) in water (78.06 g), then add talc (talc sieved through 120 mesh screen; 5 g) and mix for 10 min, the mannitol, and talc in water mixture was retained for further processing. The mannitol, and talc mixture was blended with the retained 10% HPMC mixture

### Stage 3 (Seal Coating):

Fluid bed Processor: Prewarm a fluid bed processor machine (inlet temp. 50° C. with appropriate air flow). The coated sugar spheres (100 g) were added to the fluid bed processor machine fluidization (Inlet temp.: 50° C.; Product temp.: 30-38° C. (set 40° C.); atomized air: 1.2 bar; nozzle: 1.0 mm; and spray at a speed of 1.12 mL/min) was commenced using seal coating mixture. The resulting coated material was dried at 60° C. for 1 hours in fluid bed to provide the seal coated dexlansoprazole coated sugar spheres, the product was sieved through a 30 mesh screen (bottom) and a 16 mesh screen (top). A portion of the material was retained for enteric coating.

### Stage 4 (Enteric Coating):

Eudragit L100-55 Mixture: Eudragit L100-55 (11.49 g) was mixed with isopropyl alcohol (182.16) in a suitable container. The Eudragit L100-55 mixture was retained for further processing. Talc (3.45 g) and TiO₂ (1.17 g), both sieved through a 120 mesh screen, PEG6000 (2.3 g) and Polysorbate 80 (0.53 g) were added to the Eudragit L100-55 mixture with thorough further mixing.

Fluid bed Processor (Eudragit L100-55): Prewarm a fluid bed processor machine (inlet temp. 45° C. with appropriate air flow). The seal coated dexlansoprazole coated sugar spheres (70 g) were added to the fluid bed processor machine fluidization (Inlet temp.: 45° C.; Product temp.: 32-40° C. (set 40° C.); atomized air: 1.8 bar; nozzle: 1.0 mm; and spray at a speed of 1.5 mL/min) was commenced using Eudragit L100-55 Mixture. After Eudragit L100-55 mixture layering, spray a suspension of SiO₂ (0.3 g) in isopropyl alcohol (19.63 g) on the Eudragit L100-55 coated material (60 g), the resulting material was dried at 55° C. for 1 hour in fluid bed to provide the enteric coated dexlansoprazole coated sugar spheres.

Eudragit 5100 Mixture: Eudragit 5100 (16.7 g) was mixed with isopropyl alcohol (198 g)/water (28 g) in a suitable container. The Eudragit S100 mixture was retained for further processing. TiO₂ (sieved through a 120 mesh screen; 1.31 g), triethyl citrate (TEC; 2.51 g) and Polysorbate 80 (0.5 g) were added to the Eudragit L100-55 mixture with thorough further mixing.

Fluid bed Processor: Prewarm a fluid bed processor machine (inlet temp. 45° C. with appropriate air flow). The seal coated dexlansoprazole coated sugar spheres (50 g) were added to the fluid bed processor machine, fluidization (Inlet temp.: 45° C.; Product temp.: 32-38° C. (set 40° C.); atomized air: 1.8 bar; nozzle: 1.0 mm; and spray at a speed of 2.1 mL/min) was commenced using Eudragit S100 Mixture. After Eudragit S100 mixture layering, spray a suspension of SiO₂ (0.3 g) in isopropyl alcohol (19.63 g) on the on the Eudragit S100 coated material, the resulting material was dried at 55° C. for 1 hour in fluid bed to provide the enteric coated dexlansoprazole coated sugar spheres.

Final Blend: Blend Eudragit L100-55 enteric-coated pellets (30% dexlansoprazole), Eudragit S100 enteric-coated pellets (70% dexlansoprazole) and 0.5% SiO₂ to provide the final product. The final blend was tested for impurities.

### Preparation of Sample 5:

Stage 1 (Drug Layering): Micronized calcium hydroxide (24 g) was added into a container containing acetone (426.6) and mixed for at least 30 min, then micronized dexlansoprazole (128.6) and SDS (18 g) was added and mixed for at least 45 additional minutes. The calcium hydroxide, dexlansoprazole, SDS mixture was retained for further processing. Hydroxypropylcellulose, NF (KLUCEL® EF PHARM; 67.4) was added into a container containing acetone (354.7 g) and mixed until the solid dissolved. The Hydroxypropylcellulose, NF (KLUCEL® EF PHARM) mixture was combined with the calcium hydroxide and dexlansoprazole mixture and the resulting mixture was mixed for at least 1 hour. The mixture was retained for further processing. Micronized mannitol (180 g) was added into a container containing acetone (697.6) and mixed for at least 1.5 hours. The mannitol mixture was retained for further processing. The calcium hydroxide, dexlansoprazole, SDS, Hydroxypropylcellulose, NF (KLUCEL® EF PHARM) mixture was combined with the mannitol mixture and the resulting mixture was mixed for at least 2 hours. The mixture was retained for further processing. The mixture was then sprayed onto sugar spheres (30/35 mesh; 100 g) previously placed into a fluid-bed coater equipped with bottom spraying (DPL-0.2: inlet air temperature: 31-33° C.; product temperature: 31-32° C.; coating solution spray rate: 1.77 g/min; and spray air pressure: 1 bar) to provide dexlansoprazole coated sugar spheres. The material was dried at 65° C. for 1 hour, a portion of the resulting 16-20 mesh pellets were retained for seal coating.

### Stage 2 (Preparing Seal Coating Mixture):

Mixture 1: Dissolve mannitol (8.2 g) in water (39.96 g), then add talc (talc sieved through 120 mesh screen; 2.37 g) and Ca(OH)₂ (0.77 g) and mix for 10 min, the mannitol, talc, and Ca(OH)₂ in water mixture was retained for further processing. A 10% HPMC mixture (g/g) was made by adding HPMC (1.26 g) to water and mixing, the mixture was retained for further processing. The mannitol, talc, and Ca(OH)₂ in water mixture was blended with the retained 10% HPMC mixture.

Mixture 2: Dissolve mannitol (5.82 g) in water (28.3 g), then add talc (talc sieved through 120 mesh screen; 1.68 g) and mix for 10 min, the mannitol, and talc in water mixture was retained for further processing. A 10% HPMC mixture (g/g) was made by adding HPMC (0.9 g) to water and mixing, the mixture was retained for further processing. The mannitol, and talc in water mixture was blended with the retained 10% HPMC mixture.

### Stage 3 (Seal Coating):

Fluid bed Processor Step 1: Prewarm a fluid bed processor machine (DPL-0.2: inlet temp. 45° C. with appropriate air flow). The coated sugar spheres (84 g) were added to the fluid bed processor machine fluidization (Inlet temp.: 45° C.; Product temp.: 40° C. (set 40° C.); atomized air: 1.2 bar; nozzle: 0.8 mm; and spray at a speed of 1.3 mL/min) was commenced using Mixture 1. The resulting coated material was dried at 55° C. for 0.5 hours then 65° C. for 1 hour. The resulting pellets were placed in the fluid bed processor for further coating with Mixture 2.

Fluid bed Processor Step 2: The seal coated sugar spheres (96.6 g) were added to the fluid bed processor machine fluidization (DPL-0.2: Inlet temp.: 55° C.; Product temp.: 50° C.; atomized air: 1.8 bar; nozzle: 0.8 mm; and spray at a speed of 0.6 mL/min) was commenced using Mixture 2. The resulting coated material was dried at 55° C. for 0.5 hours then 65° C. for 1 hour to provide the seal coated dexlansoprazole coated sugar spheres, the product was sieved through a 30 mesh screen (bottom) and a 16 mesh screen (top). A portion of the material was retained for enteric coating.

### Stage 4 (Enteric Coating):

Eudragit L100-55 Mixture: Eudragit L100-55 (4.54 g) was mixed with isopropyl alcohol (99 g) in a suitable container. The Eudragit L100-55 mixture was retained for further processing. Talc (0.99 g), triethyl citrate (TEC; 0.59 g) and Polysorbate 80 (0.23 g) were added to the Eudragit L100-55 mixture with thorough further mixing for 30 min.

Fluid bed Processor (Eudragit L100-55): Prewarm a fluid bed processor machine (DPL-0.2: inlet temp. 45° C. with appropriate air flow). The seal coated dexlansoprazole coated sugar spheres (45 g) were added to the fluid bed processor machine fluidization (Inlet temp.: 45° C.; Product temp.: 40° C.; atomized air: 1.6 bar; nozzle: 0.8 mm; and spray at a speed of 1.2 mL/min) was commenced using Eudragit L100-55 Mixture. After Eudragit L100-55 mixture layering, a suspension of SiO₂ (0.26 g) in isopropyl alcohol (19.74 g) was sprayed on the Eudragit L100-55 coated material (51.75 g), the resulting material was dried at 60° C. for 1 hour in an oven to provide the enteric-coated dexlansoprazole sugar spheres.

Eudragit 5100 Mixture: Eudragit 5100 (10.32 g) was mixed with isopropyl alcohol (122.75 g)/water (17.33 g) in a suitable container. The Eudragit 5100 mixture was retained for further processing. Triethyl citrate (TEC; 1.56 g) and Polysorbate 80 (0.27 g) were added to the Eudragit 5100 mixture with through further mixing for 15 min.

Fluid bed Processor: Prewarm a fluid bed processor machine (inlet temp. 45° C. with appropriate air flow). The seal coated dexlansoprazole coated sugar spheres (45 g) were added to the fluid bed processor machine, fluidization (DPL-0.2: Inlet temp.: 45° C.; Product temp.: 40° C.; atomized air: 1.8 bar; nozzle: 0.8 mm; and spray at a speed of −2 mL/min) was commenced using Eudragit S100 Mixture. After Eudragit S100 mixture layering, a suspension of SiO₂ (0.29 g) in isopropyl alcohol (19.71 g) was sprayed on the Eudragit 5100 coated material (58.5 g), the resulting material was dried at 60° C. for 1 hour in an oven to provide the enteric-coated dexlansoprazole sugar spheres.

Final Blend: Blend Eudragit L100-55 enteric-coated pellets (30% dexlansoprazole), Eudragit S100 enteric-coated pellets (70% dexlansoprazole) and 0.5% SiO₂ to provide the final product. The final blend was tested for impurities.

### Preparation of Sample 6:

Stage 1 (Drug Layering): Micronized calcium hydroxide (0.906 kg) was added into a container containing acetone (42 kg) and mixed for at least 30 min, subsequently micronized dexlansoprazole (4.530 kg) and micronized SDS (0.452 kg) was added and mixed for at least 45 additional minutes. The calcium hydroxide, dexlansoprazole, and SDS mixture was then treated with micronized mannitol (6.794 kg) and mixed for at least an additional 15 min. The mixture was retained for further processing. Hydroxypropylcellulose, NF (KLUCEL® EF PHARM; 2.544 kg) was added into a container containing acetone (13 kg) and mixed until the solid dissolved. The Hydroxypropylcellulose, NF (KLUCEL® EF PHARM) mixture was combined with the calcium hydroxide, dexlansoprazole, SDS, and mannitol mixture and the resulting mixture was mixed for at least 1 hour. The mixture was retained for further processing. The calcium hydroxide, dexlansoprazole, SDS, mannitol, and Hydroxypropylcellulose mixture was retained for further processing. The mixture was then sprayed onto sugar spheres (30/35 mesh; 3.773 kg) previously placed into a fluid-bed coater equipped with a 12″ Wurster insert (inlet air temperature: 33-38° C.; product temperature: 24-26° C.; coating solution spray rate: 50-150 g/min; air volume 165-220 cfm; and spray air pressure: 1 bar (range 0.8-1.2); Wurster nozzle: 4.0 mm port) to provide dexlansoprazole coated sugar spheres. The material was dried at 70° C. for 1 hour, the resulting 16-20 mesh pellets were retained for seal coating.

### Stage 2 (Preparing Seal Coating Mixture):

Mixture 1: Combine Ca(OH)₂ (0.099 kg) with water (6.753 kg) and mixed for 30 min, followed by addition of mannitol (1.05 kg) with 15 min mixing, then Hydroxypropylcellulose, NF (KLUCEL® EF PHARM; 0.163 kg) with at least 45 min mixing and finally addition of talc (0.304 kg) with 30 min mixing. The Ca(OH)₂, mannitol, Klucel EF Pharm, and talc in water mixture was retained for further processing.

Mixture 2: Opadry Clear (1.53 kg) was combined with water (11.26 kg) and mixed for at least 30 min. The Opadry Clear in water mixture was retained for further processing.

### Stage 3 (Seal Coating):

Fluid bed Processor Step 1: Prewarm a fluid bed processor machine (Glatt GPCG-15 with 12″ Wurster insert with inlet temp. 50° C. with appropriate air flow). The coated sugar spheres (16.15 kg) were added to the fluid bed processor machine, fluidization (Inlet temp.: 63-78° C.; Product temp.: 38-43° C.; atomized air: 1.5 bar; Wurster nozzle: 4.0 mm port; process air volume 400-480 cfm; and spray rate: 50-150 g/min) was commenced using Mixture 1. The resulting coated material was dried at 65-72° C. for 60 min. to provide 14-60 mesh pellets. The resulting pellets were placed in the fluid bed processor for further coating with Mixture 2.

Fluid bed Processor Step 2: Prewarm a fluid bed processor machine (Glatt GPCG-15 with 12″ Wurster insert with inlet temp. 70° C. with appropriate air flow). The Mixture 1 coated sugar spheres (16.88 kg) were added to the fluid bed processor machine, fluidization (Inlet temp.: 65-85° C.; Product temp.: 44-52° C.; atomized air: 1.5-2.0 bar; Wurster nozzle: 4.0 mm port; process air volume 400-480 cfm; and spray rate: 50-125 g/min) was commenced using Mixture 2. The resulting coated material was dried at 58-62° C. for 60 min. The 14-60 mesh pellets were retained for enteric coating.

### Stage 4 (Enteric Coating):

Eudragit L100-55 Mixture: Eudragit L100-55 (1.086 kg) was mixed for 5 min. with isopropyl alcohol (21.77 kg) in a suitable container, then triethyl citrate (TEC; 0.13 kg) was added with mixing for at least 5 min, followed by Polysorbate 80 (0.051 kg) with mixing for at least 5 min and finally talc (0.218 kg) with mixing for art least 5 min. The Eudragit L100-55 mixture was retained for further processing.

Fluid bed Processor (Eudragit L100-55): Prewarm a fluid bed processor machine (Glatt GPCG-15 with 12″ Wurster insert with 350 cfm to achieve product temp of 36-40° C.). The seal coated dexlansoprazole coated sugar spheres (5.5 kg) were added to the fluid bed processor machine, fluidization (Inlet temp.: 51-60° C.; Product temp.: 36-40° C.; atomized air: 2.0 bar; Wurster nozzle: 4.0 mm port; process air volume 200-400 cfm; and spray rate: 50-150 g/min) was commenced using Eudragit L100-55 Mixture. The pellets were dried for 3 hours at product temp of 50-55° C. to provide 14-60 mesh as final L100-55 coated pellets.

Eudragit S100 Mixture: Eudragit 5100 (1.245 kg) was mixed with isopropyl alcohol (14.629 kg)/water (2.091 kg) in a suitable container, then triethyl citrate (TEC; 0.374 kg) was added with mixing for at least 5 min, followed by Polysorbate 80 (0.033 kg) with mixing for at least 5 min and finally talc (0.163 kg) with mixing for art least 5 min. The Eudragit 5100 mixture was retained for further processing.

Fluid bed Processor (Eudragit S100): Prewarm a fluid bed processor machine (Glatt GPCG-15 with 12″ Wurster insert with 350 cfm to achieve product temp of 40° C.). The seal coated dexlansoprazole coated sugar spheres (5.5 kg) were added to the fluid bed processor machine, fluidization (Inlet temp.: 55-62° C.; Product temp.: 40-42° C.; atomized air: 2.0 bar; Wurster nozzle: 4.0 mm port; process air volume 200-300 cfm; and spray rate: 50-100 g/min) was commenced using Eudragit S100 Mixture. The pellets were dried for 3 hours at product temp of 65-70° C. to provide 14-60 mesh as final S100 coated pellets.

The final L100-55 enteric coated pellets and final 5100 enteric coated pellets were tested for impurities separately.

### Example 5

### Dexlansoprazole Containing Pellets

### Sample Preparation

To a container containing acetone (9.38 g) was added micronized calcium hydroxide (0.2 g) with mixing for at least 0.5 h. To the mixing dispersion was added micronized dexlansoprazole (1 g), water (0.07 g) and sodium dodecyl sulfate (SDS) (0.033 g), the resulting mixture was mixed for at least 10 minutes. Subsequently, micronized mannitol (1.5 g) was added to the container and further mixing was continued for at least 1 h.

To a separate container containing acetone (2.94 g) was added hydroxypropylcellulose (0.56 g; NF (KLUCEL® EF PHARM)), with mixing (continued until all solids dissolved).

The resulting mixture was transferred into the initial mixture with mixing, continued for at least 1 hour.

The mixture was then applied to a clean glass plate and dried. The recovered material from the glass plate was grinded into powder and used for measuring the melting point.

### Example 6

### Formulation of Dexlansoprazole and Calcium Hydroxide

Dexlansoprazole, and Ca(OH)₂, were mixed in acetone. The mixture was dried on a glass plate at room temperature to provide a solid. The X-ray diffraction pattern of the formulation exhibits a 28 peak at 4.53, and 5.28. Source: Cu (40 kV, 250 mA). Wavelength to compute d-spacing=1.54059 (Cu/K-alpha1).

### Example 7

### Formulation of Dexlansoprazole Including Layering on Sucrose Spheres Sample Preparation

To a container containing acetone (42 kg) was added calcium hydroxide (0.906 kg) with mixing for 0.5 h. To the mixture was added dexlansoprazole (4.53 kg), and sodium dodecyl sulfate (SDS) (0.452 kg). The resulting mixture was mixed for at least 45 minutes. Subsequently, mannitol (6.794 kg) was added to the container and further mixing was continued for at least 1.5 h, which yielded a first mixture.

To a separate container containing acetone (13 kg) was added hydroxypropylcellulose (2.544 kg; NF (KLUCEL® EF PHARM). The resulting mixture was mixed for at least 1 h, which yielded a second mixture.

The second mixture was transferred into the first mixture with mixing, continued for 4 h to provide the final mixture for coating sugar spheres.

Sugar spheres (3.773 kg; 30-35 mesh) were added to a fluid bed. The sugar spheres were sprayed with the final mixture for coating the spheres then dried for 1 h to afford 17.2 kg of dexlansoprazole containing pellets.

1. A process for preparing a dexlansoprazole composition comprising:
preparing a first mixture by mixing dexlansoprazole, a base, a sugar alcohol, and a first excipient in a mixture of water and an organic solvent; and drying to provide a dexlansoprazole composition, wherein the base is neither MgO nor MgCO₃.

2. (canceled)

3. The process of claim 1, wherein the base includes a Ca²⁺ counterion.

4. (canceled)

5. (canceled)

6. The process of claim 1, wherein the base is selected from the group consisting of Ca(OH)₂, CaO, a mixture of CaCO₃ and NaOH, and mixtures thereof.

7. The process of claim 1, wherein the base is Ca(OH)₂.

8. The process of claim 1, wherein the organic solvent is selected from the group consisting of acetone, ethyl acetate, ethyl alcohol and mixtures thereof.

9. The process of claim 1, wherein the sugar alcohol is mannitol.

10. The process of claim 1, wherein the first excipient is hydroxypropylcellulose.

11. (canceled)

12. The process of claim 1,
wherein:
the base is Ca(OH)₂;
the sugar alcohol is mannitol;
the first excipient is hydroxypropylcellulose; and
the solvent is a mixture of water and an organic solvent.

13. (canceled)

14. (canceled)

15. (canceled)

16. The process of claim 1, wherein the drying includes spray drying, and drying under reduced pressure, wherein the drying occurs at, below or above room temperature.

17. A dexlansoprazole formulation comprising:
the composition formed by the process of claim 1 and, in addition, a pharmaceutically acceptable excipient.

18. (canceled)

19. (canceled)

20. A dexlansoprazole formulation comprising:
dexlansoprazole, a base, and a sugar alcohol, wherein the base is selected from the group consisting of Ca(OH)₂, CaO, a mixture of CaCO₃ and NaOH, and mixtures thereof.

21. The formulation of claim 20, wherein the base is Ca(OH)₂.

22. The formulation of claim 20, wherein the sugar alcohol is mannitol.

23. The formulation of claim 20, wherein:
the base is Ca(OH)₂;
the sugar alcohol is mannitol; and further comprises
low substitution hydroxypropylcellulose, and sucrose spheres.

24. The formulation of claim 20, wherein dexlansoprazole is in the form of a salt or hydrate.

25. (canceled)

26. (canceled)

27. (canceled)

28. (canceled)

29. A dexlansoprazole formulation comprising:
dexlansoprazole, and Ca(OH)₂.

30. (canceled)

31. A method of treating or preventing a digestive disorder in a mammal thereof comprising administering to said mammal an effective amount of a formulation of claim 20.

32. A process for preparing the dexlansoprazole formulation of claim 29 comprising:
preparing a mixture by mixing dexlansoprazole and Ca(OH)₂ in a mixture of water and an organic solvent; and drying to provide the dexlansoprazole composition.

33. The process of claim 32 further comprising:
layering the mixture on a support matrix to provide a coated excipient mixture,
wherein the drying comprises drying the coated excipient mixture to provide the dexlansoprazole composition.

34. The process of claim 32, wherein the organic solvent is acetone.
